(19) United States
(12) Patent Application Publication
(10) Pub. No.: US 2016/0205925 A1
(43) Pub. Date: Jul. 21, 2016

(54) ANTIMICROBIAL COMPOSITIONS MADE OF A THERMOPLASTIC POLYMER AND A PHOTOSENSITIZER

(71) Applicant: Ariel-University Research and Development Company Ltd., Ariel (IL)

(72) Inventors: Marina NISNEVITCH, Nofim (IL); Faina NAKONECHNY, Ariel (IL); Anton VALKOV, Ariel (IL)

(21) Appl. No.: 14/995,414

(22) Filed: Jan. 14, 2016

Related U.S. Application Data
(60) Provisional application No. 62/103,703, filed on Jan. 15, 2015.

(51) Int. Cl. A01N 25/10; A61K 41/00; A01N 43/90; A61N 5/06; A01N 43/84; C02F 1/72; C02F 1/32; A61L 2/08; A01N 43/16

(52) U.S. Cl. CPC A01N 25/10; A61L 2/088; A61K 41/0057; A01N 43/90; A01N 43/16; A01N 43/84; C02F 1/725; C02F 1/32; A61N 5/062; C02F 2303/04; C02F 2305/10

(57) ABSTRACT

Compositions comprising a polymeric matrix made of a thermoplastic polymer and a photosensitizer incorporated in the polymeric matrix are provided. Also provided are processes of preparing the compositions, without using an organic solvent or a surfactant. Articles-of-manufacturing comprising the compositions, which can be self-disinfecting, and uses of the composition or articles-of-manufacturing comprising same for disinfecting bodily or inanimate substrates such as water are also provided.

### RELATED APPLICATION

This application claims the benefit of priority under 35 USC 119(e) of U.S. Provisional Patent Application No. 62/103,703 filed on Jan. 15, 2015, the contents of which are incorporated herein by reference in their entirety.

### FIELD AND BACKGROUND OF THE INVENTION

The present invention, in some embodiments thereof, relates to material chemistry, and more particularly, but not exclusively, to photosensitive compositions and articles-of-manufacturing containing same which exhibit antimicrobial activity upon exposure to light, to processes of preparing same and uses thereof.

A photosensitizer (PS) is a chemical entity, which, upon absorption of light, induces a chemical or physical alteration of another chemical entity. Photosensitizers typically act by absorbing energy from light to enter an excited state, and in the excited state interact with another chemical entity and transfer the energy thereto. The excited PS can follow two pathways, named Type I and Type II reactions. In the Type I mechanism, PS molecules react directly with (e.g., bio-organic) molecules to produce active free radicals and radical ions. The Type II reaction is accompanied by an energy transfer to molecular oxygen in an oxygen-containing environment (e.g., oxygen dissolved in an aqueous phase). The photosensitizer typically interacts with triplet oxygen species to produce reactive oxygen species (ROS) such as singlet oxygen species. Photosensitizers that produce ROS from triplet oxygen are also referred to in the art as triplet photosensitizers.

Singlet oxygen is generated in neutrophils and macrophages for use in killing microorganisms. Superoxide dismutases, catalases, and peroxidases are defenses against radical- and reduced-oxygen species, but are not effective against singlet oxygen. A few microorganisms, such as Cercospora, are inherently resistant to singlet oxygen, and Gram-positive bacteria are generally more easily killed by singlet oxygen than Gram-negative bacteria. Enveloped viruses are inactivated by singlet oxygen more readily than non-enveloped viruses. Acquired resistance by a bacterium, fungus, or virus to singlet oxygen has not been reported to date.

Chemical attachment (e.g., covalent or ionic), or physical inclusion, of PSs to or in solid supports has been recognized as advantageous over the use PSs in solution. Immobilized PSs can be used in solvents in which free PSs are insoluble, they can be easily removed at the end of the treatment, can be reused, can be introduced into continuous processes, and are more resistant to bleaching by light and oxygen than free PSs.

Immobilization of photosensitizers has been performed by covalent bonding and adsorption onto a solid support, formation of ionic bonds between ion-exchange resins and photosensitizers or incorporation of the photosensitizers into polymeric films. It was demonstrated that application of these methods of immobilization leads to the production of samples with good photodynamic properties which are retained for a long period of time.

Covalent attachment of several PSs, such as Rose Bengal (RB), eosin, fluorescein, chlorophyllin, hematoporphyrin and Zn(II) phthalocyanine tetrasulfonic acid to various supports, including silica gel, poly(styrene-co-vinylbenzyl chloride), poly [N-isopropylacrylamide)-co-(vinylbenzyl chloride)], poly[(sodium p-styrenesulfonate)-co-(4-vinylbenzyl chloride)] and chitosan has been reported. PSs that were covalently attached to polymers demonstrated high (up to 0.91) quantum yields of singlet oxygen formation.

U.S. Pat. No. 5,830,526 describes an incorporation of xanthenes dyes like Rose Bengal, into polymer matrices by forming a woven or nonwoven fabric having a non-leachable light-activated dye bound thereto by means of a cationic or anionic binder such as a water soluble polymer or carrageenan.

Bezman et al. [Photochemistry and Photobiology, 28, 325-329; 1978] disclose the photodynamic inactivation of E. coli by Rose Bengal immobilized on polystyrene beads.

U.S. Pat. No. 6,420,455 discloses hardened polymer compositions that possess antimicrobial activity, in both the light and the dark, comprising polymers and one or more photosensitizers, at least one of which is a xanthene photosensitizer (Rose Bengal, erythrosin, eosin yellowish, fluorescein), wherein the photosensitizer is not bound to the polymer through covalent interactions. The antimicrobial compositions are incorporated in various self-supporting films, coating surfaces and medical articles.

WO 1993/000815 discloses photobactericidal and autosterile compositions comprising a polymer and a photosensitizer, used in a method of sterilizing a surface containing such a photosensitizer by exposing the surface to visible light. The photosensitizer is a porphyrin or a phthalocyanine, more particularly a salt of the meso-tetra(N-alkyl-4-pyridinium)porphyrin tetracation. The composition is formed either by dyeing the polymer with a solution of the photosensitizer or by including the photosensitizer in a melt or dope of the polymer which is then formed into a solid article (e.g., fiber, textile article).

WO 1999/049823 discloses antimicrobial, light sensitive, laminar structures for food packaging wraps and medical equipment, which contain a photosensitizer, preferably methylene blue, dispersed therein. The materials may be made by cryogenically grinding together the photosensitizer, a surfactant material, and a polymer resin, to form a uniform concentrate as a homogeneous fine powder. The concentrate is added to large batches of polymer that are processed to form plastic films, melt-blown nonwovens, fabrics, and other formed articles.

U.S. Pat. Nos. 5,709,994 and 5,618,732 disclose compositions comprising a photosensitizer and a chemiluminescent compound capable of being activated by singlet oxygen, incorporated in a solid or fluid particulate matrix or a non-particulate polymeric matrix by means of covalent bonding or dissolution. The compositions are used in methods for labeling a material (e.g., an analyte) by means of delayed luminescence, which can be realized upon irradiation of the matrix (the amount of luminescence generated is related to the amount of the analyte in a medium).

U.S. Pat. No. 7,449,194 discloses a polymeric body covering article, more particularly gloves, for reducing microbiological contamination, wherein the article generates and releases gas upon activation by electromagnetic energy and/or moisture, whereby the released gas provides antimicrobial and/or anti odor protection to objects in contact or in proximity to the interior and/or exterior surface of the article. Photosensitizers are optional components in the composition, introduced in order to shift the absorption wavelength of the composition, particularly a shift to visible absorption wavelength, to thereby improve activation by room lightning.

EP Patent No. 215332 discloses a colored thermoplastic composition prepared by incorporating the colorant into the resin in a molten or pelletized state whereby the colorant is a polyalkyleneoxy substituted chromophore, which is not chemically bound to the resin. The resin is preferably a polyolefin polymer such as linear low density polyethylene, polypropylene, polybutylene and copolymers made from ethylene, propylene and/or butylene.

U.S. Patent Application Publication No. 2004/0197300 discloses a composition for environmental clarification having active oxygen generating action, which comprises, as the active oxygen generating agent, polyaniline compounded or mixed with calcium phosphate, made by forming a porous calcium phosphate coating on at least part of the surface of polyaniline particles. Active oxygen is generated by bringing a liquid that contains dissolved oxygen in contact with the active oxygen generating agent.

Nakonechny et al. [Photochemistry and Photobiology, 2013, 89: 671-678] describes the inclusion of PSs in a polymeric film by mixing chloroform solutions of the PSs Methylene blue (MB) and Rose Bengal (RB), with polystyrene solutions in the samesolvent with subsequent air evaporation of the latter.

Valkov et al. [Applied Mechanics and Materials Vols. 719-720 (2015) pp 21-24] shows that Rose Bengal incorporated in polystyrene, polycarbonate and poly(methyl methacrylate) eradicates Gram-positive Staphylococcus aureus bacteria under moderate illumination.

### SUMMARY OF THE INVENTION

The present inventors have explored methodologies for incorporating a photosensitizer into polymeric compositions and have surprisingly uncovered that such polymeric compositions can be prepared from a mixture of a thermoplastic polymer and a photosensitizer, without using organic solvents or any other additional solubilizers (such as, for example, surfactants). The present inventors have demonstrated that heating a mixture containing a thermoplastic polymer and a photosensitizer at a temperature that is equal to or higher than a Tm of the polymer results in compositions which substantially incorporate the photosensitizer within the polymeric matrix formed from the polymer, and which are characterized by low leaching of the photosensitizer from the polymeric matrix, by high photostability and by high antimicrobial activity. The methodologies presented herein are particularly useful for incorporating a photosensitizer in non-soluble thermoplastic polymers.

According to an aspect of some embodiments of the present invention there is provided a composition comprising a polymeric matrix comprised of a thermoplastic polymer and a photosensitizer incorporated in the polymeric matrix, the composition being devoid of a surfactant and/or a residual amount of an organic solvent.

According to some of any of the embodiments of the present invention, the polymer matrix consists essentially of the thermoplastic polymer and the photosensitizer.

According to some of any of the embodiments of the present invention, the composition consists essentially of the thermoplastic polymer and the photosensitizer.

According to some of any of the embodiments of the present invention, the thermoplastic polymer has a Tm value lower than a temperature at which the photosensitizer decomposes.

According to some of any of the embodiments of the present invention, the thermoplastic polymer is a non-soluble thermoplastic polymer.

According to some of any of the embodiments of the present invention, the non-soluble thermoplastic polymer is a polyolefin.

According to some of any of the embodiments of the present invention, the thermoplastic polymer is selected from the group consisting of polyethylene, poplypropylene, poly(vinyl chloride), poly(vinylidene chloride), polytetrafluoroethylene, poly(methylmethacrylate), poly(vinyl acetate), cis-polyisoprene and polychloroprene.

According to some of any of the embodiments of the present invention, the photosensitizer is selected from the group consisting of a xanthene dye, a porphyrin, a phenothiazine dye, a triphenylmethine dye, an oxazine dye, a phthalocyanine, a psoralene and a perylenequinonoid.

According to some of any of the embodiments of the present invention, the photosensitizer is xanthene dye.

According to some of any of the embodiments of the present invention, an amount of the one or more photo sensitizers is in the range of from about 0.1 weight percent to about 10 weight percents of the total weight of the composition-of-matter.

According to some of any of the embodiments of the present invention, less than 20% of the photosensitizer is present on an external surface of the polymeric matrix.

According to some of any of the embodiments of the present invention, the composition is such that upon immersing 2 grams of the composition in 2 liters of an aqueous solution for 24 hours, less than 5% of the photosensitizer leaches out of the composition.

According to some of any of the embodiments of the present invention, the composition is such that upon exposure to illumination of a luminescent lamp for 10 days, the photosensitizer exhibits at least 80% of its photosensitizing activity.

According to an aspect of some embodiments of the present invention there is provided a process of preparing the composition as described herein, the process comprising:

heating a solid mixture comprising the photosensitizer and the thermoplastic polymer at a temperature equal to or higher than a Tm of the polymer, to thereby obtain a fluid mixture of the photosensitizer and the polymer; and

cooling the fluid mixture to a temperature below the Tm of the polymer, thereby obtaining the composition.

According to some of any of the embodiments of the present invention, the process further comprises mixing the mixture, during the heating.

According to some of any of the embodiments of the present invention, the heating and the mixing are performed in an extruder.

According to some of any of the embodiments of the present invention, the solid mixture and the fluid mixture are devoid of an organic solvent and/or a surfactant.

According to some of any of the embodiments of the present invention, the solid mixture and the fluid mixture consist essentially of the polymer and the photo sensitizer.

According to some of any of the embodiments of the present invention, a weight ratio of the polymer and the photosensitizer in the solid mixture ranges from 1000:1 to 10:1.

According to some of any of the embodiments of the present invention, the process further comprises, subsequent to the heating, subjecting the fluid mixture or the composition to a processing procedure.

According to an aspect of some embodiments of the present invention there is provided a process of preparing an antimicrobial composition, the process comprising heating a solid mixture comprising a thermoplastic polymer and a photosensitizer at a temperature equal to or higher than a Tm of the polymer, to thereby obtain a fluid mixture of the photosensitizer and the polymer; and cooling the fluid mixture, to thereby obtain the antimicrobial composition.

According to some of any of the embodiments of the present invention, the Tm of the polymer is lower than a temperature at which the photosensitizer decomposes.

According to some of any of the embodiments of the present invention, the process further comprises mixing the mixture, during the heating.

According to some of any of the embodiments of the present invention, the heating and the mixing are performed in an extruder.

According to some of any of the embodiments of the present invention, the solid mixture and the fluid mixture are devoid of an organic solvent and/or a surfactant.

According to some of any of the embodiments of the present invention, the solid mixture and the fluid mixture consist essentially of the polymer and the photo sensitizer.

According to some of any of the embodiments of the present invention, a weight ratio of the polymer and the photosensitizer in the solid mixture ranges from 1000:1 to 10:1.

According to some of any of the embodiments of the present invention, the process further comprises, subsequent to the heating, subjecting the fluid mixture or the composition to a processing procedure.

According to some of any of the embodiments of the present invention, the thermoplastic polymer is a non-soluble thermoplastic polymer, as described herein.

According to some of any of the embodiments of the present invention, the photosensitizer is selected from the group consisting of a xanthene dye, a porphyrin, a phenothiazine dye, a triphenylmethine dye, an oxazine dye, a phthalocyanine, a psoralene and a perylenequinonoid.

According to some of any of the embodiments of the present invention, the photosensitizer is a xanthene dye.

According to an aspect of some embodiments of the present invention there is provided a composition, e.g., an antimicrobial composition, prepared by a process as described in any one of the process embodiments herein and any combinations thereof.

According to some of any of the embodiments of the present invention, the antimicrobial composition is devoid of a surfactant and/or a residual amount of an organic solvent.

According to some of any of the embodiments of the present invention, an amount of the one or more photo sensitizers is in the range of from about 0.1 weight percent to about 10 weight percents of the total weight of the composition-of-matter.

According to some of any of the embodiments of the present invention, less than 20% of the photosensitizer is present on an external surface of the polymeric matrix.

According to some of any of the embodiments of the present invention, the composition is such that upon immersing 2 grams of the composition in 2 liters of an aqueous solution for 24 hours, less than 5% of the photosensitizer leaches out of the composition.

According to some of any of the embodiments of the present invention, the composition is such that upon exposure to illumination of a luminescent lamp for 10 days, the photosensitizer exhibits at least 80% of its photosensitizing activity.

According to an aspect of some embodiments of the present invention there is provided an article-of-manufacturing comprising a composition (e.g., an antimicrobial composition), as described in any of the composition embodiments and any combination thereof.

According to an aspect of some embodiments of the present invention there is provided a method of disinfecting or sterilizing the composition as described herein (e.g., a antimicrobial composition) or the article-of-manufacturing as described herein in any one of its respective embodiments, and any combination thereof, the method comprising exposing the composition or the article-of-manufacturing to conditions in which the photosensitizer generates reactive oxygen species.

According to an aspect of some embodiments of the present invention there is provided a method of disinfecting a substrate, the method comprising contacting the substrate with a composition as described herein (e.g., a antimicrobial composition) or the article-of-manufacturing as described herein in any one of its respective embodiments, and exposing the composition or the article-of-manufacturing to conditions in which the photosensitizer generates reactive oxygen species.

According to some of any of the embodiments of the present invention, the substrate harbors, or is suspected as harboring, a microorganism.

According to some of any of the embodiments of the present invention, the substrate is a bodily tissue, fluid or cavity.

According to some of any of the embodiments of the present invention, the composition or article-of-manufacturing is a medical device.

According to some of any of the embodiments of the present invention, the method is for treating a medical condition associated with a microorganism in a subject in need thereof.

According to some of any of the embodiments of the present invention, the substrate is an inanimate substrate.

According to some of any of the embodiments of the present invention, the substrate comprises water and the method is for disinfecting the water.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE OF THE SEVERAL VIEWS OF THE DRAWINGS

The patent or application file contains at least one drawing executed in color. Copies of this patent or patent application publication with color drawing(s) will be provided by the Office upon request and payment of the necessary fee.

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 presents the TGA data of PE (black), RBS (green) and PE-RBS (PE having RBS incorporated therein) (blue). Upper diagram presents mass changes and lower diagram presents specific power changes;

FIGS. 2A-C present SEM images of PE alone before extrusion (FIG. 2A) and of PE-RBS after co-extrusion (FIGS. 2B and 2C, cross-sectional and surface view, respectively);

FIGS. 3A-D present EDS data of PE before extrusion (FIG. 3A), RBS alone (FIG. 3B), and PE-RBS after co-extrusion (FIGS. 3C and 3D showing the cross-section and surface, respectively);

FIG. 4 presents the TGA data of PE (black), MB (blue) and PE-MB after co-extrusion (red). The upper diagram presents mass changes and lower diagram presents specific power changes;

FIGS. 5A-B present SEM images of PE-MB after co-extrusion (FIGS. 5A and 5B, cross-sectional and external surface view, respectively);

FIGS. 6A-C present EDS data of MB alone (FIG. 6A), and of the cross-section (FIG. 6B) and surface (FIG. 6C) of the PE-MB after co-extrusion;

FIG. 7 presents the TGA data of PP (black), RBS (blue) and PP-RBS after co-extrusion (red). The upper diagram presents mass changes and the lower diagram presents specific power changes;

FIGS. 8A-C present SEM images of PP alone (FIG. 8A) and of PP-RBS after co-extrusion (FIGS. 8B and 8C, cross-sectional and surface view, respectively);

FIGS. 9A-C present EDS data of PP alone (FIG. 9A) and of the cross-section (FIG. 9B) and surface (FIG. 9C) of a PP-RBS composition after co-extrusion;

FIG. 10 presents images of exemplary rod prepared by extruding a mixture of PE and RBS (upper image) and of pellets obtained upon chopping the rod (lower image);

FIGS. 11A-C are bar graphs presenting the antibacterial activity of a PE-RBS composition against Gram-positive S. aureus (FIG. 11A), Gram-negative E. coli (FIG. 11B) and Gram-positive Streptococcus (FIG. 11C) in a batch regime;

FIGS. 12A-B are bar graphs presenting the antibacterial activity of a PP-RBS composition against Gram-positive S. aureus (FIG. 12A) and Gram-negative E. coli (FIG. 12B) in a batch regime;

FIG. 13 presents comparative plots showing the antibacterial activity of a PE-RBS composition against Gram-positive S. aureus in a continuous regime compared to control;

FIGS. 14A-B are bar graphs showing the effect of various concentrations of PE-RB beads (FIG. 14A) and PE-MB beads (FIG. 14B) on the antibacterial activity against S. aureus;

FIGS. 15A-B are bar graphs showing the antibacterial activity of PE-RB beads against various concentrations of S. aureus (FIG. 15A) and Streptococcus (FIG. 15B);

FIGS. 16A-D present comparative plots showing the effect of illumination intensity on the antibacterial activity of PE-RB against Streptococcus at 1.25 mW/cm² illumination (FIG. 16A) and at 6.04 mW/cm² illumination (FIG. 16B), and of PE-MB against P. aeruginosa at 1.25 mW/cm² illumination (FIG. 16C) and at 11.6 mW/cm² illumination (FIG. 16D);

FIGS. 17A-C are bar graphs showing the effect of PS loading on the antibacterial activity of PE-RB (0.2%, FIG. 17A, and 1%, FIG. 17B) and PE-MB (0.2% and 1%, FIG. 17C) against S. aureus (FIGS. 17A and B) and P. aeruginosa (FIG. 17C); and

FIG. 18 presents comparative plots demonstrating the photostability of a PP-RBS composition compared to control.

### DESCRIPTION OF SPECIFIC EMBODIMENTS OF THE INVENTION

The present invention, in some embodiments thereof, relates to material chemistry, and more particularly, but not exclusively, to photosensitive compositions and articles-of-manufacturing containing same which exhibit antimicrobial activity upon exposure to light, to processes of preparing same and uses thereof.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

As discussed in the Background section hereinabove, triplet photosensitizers exhibit a beneficial antimicrobial activity, and are even more advantageous when used in an immobilized form thereof. Immobilization of triplet photosensitizers has been performed, heretofore, by means of covalent attachment to a polymeric matrix or physical inclusion in a polymeric matrix. Chemical and physical inclusion in a polymeric matrix typically requires a use of chemicals or organic solvents which are toxic, expensive and/or environmental unfriendly if not hazardous.

The present inventors have developed a simple and cost effective, yet highly efficient, methodology for the inclusion of triplet photosensitizers in a polymeric matrix. This methodology is particularly advantageous for inclusion of the photosensitizers in polymeric matrices made of polymers which are non-soluble in water and in organic solvents, and are thermoplastic, such as polyolefins.

Using this methodology, the present inventors have prepared various compositions, made of various photosensitizers and various non-soluble thermoplastic polymers, and have shown that these compositions are stable both in terms of leaching of the photosensitizer from the polymeric matrix, and in terms of photostability. These compositions exhibit high antimicrobial activity when exposed to light in an oxygen-containing environment, without losing the antimicrobial activity for at least 10 days.

Embodiments of the present invention therefore relate to compositions comprising a photosensitizer incorporated in a polymer matrix made of a thermoplastic polymer; to a process of preparing same and to uses thereof as antimicrobial and/or self-disinfecting compositions, in manufacturing antimicrobial and/or self-disinfecting articles, and in disinfecting bodily substrates as well as inanimate substrates.

The Photosensitizer:

Herein throughout, and with reference to any one of the embodiments described herein and any combination thereof, the term “photosensitizer”, which is abbreviated as PS, describes a compound which, upon absorption of light, enters, or transforms to, an excited state, and in the excited state interacts with another chemical entity and transfers energy thereto.

In preferred embodiments of the present invention, the PS is such that interacts with molecular oxygen so as to generate reactive oxygen species such as singlet oxygen, as these terms (reactive oxygen species, ROS, and singlet oxygen) are known in the art. As discussed hereinabove and is known in the art, PSs which generate reactive oxygen species are also known as exhibiting an antimicrobial activity, as defined herein.

Thus, in some embodiments, a photosensitizer as described herein is such that generates reactive oxygen species upon exposure to light in an oxygen-containing environment (e.g., air, water, bodily fluids, and any other oxygen-containing fluids).

The photosensitizing activity of a photosensitizer can therefore be determined by a generation of reactive oxygen species, which in turn, can be determined by determining antimicrobial activity of a photosensitizer or a composition comprising the photosensitizer.

Any suitable photosensitizer may be used in conjunction with the present embodiments, so long as it exhibits antimicrobial activity as defined herein when exposed to light (illumination). Combinations of photosensitizers may also be employed, for example, for broadening the spectrum of the antimicrobial activity.

In some embodiments, the photosensitizer absorbs light at a wavelength within the continuous electromagnetic spectrum such within the ultraviolet (“UV”) range, the visible light range, and/or the infrared (near, mid and far) range, etc. For example, the absorbed light wavelengths can be between about 160 nm to about 1600 nm, between 400 nm to about 800 nm, between about 500 nm to about 850 nm, or between about 500 nm to about 700 nm, although the wavelengths may vary depending upon the particular photosensitizer(s) used.

In some embodiments, the photosensitizer absorbs light at a wavelength within the visible light, typically in a range of from 350 nm to 1200 nm, or from 400-900 nm

Exemplary photosensitizers include, without limitation, porphyrins (e.g. haematoporphyrin derivatives, deuteroporphyrin), phthalocyanines (e.g. zinc, silicon and aluminium phthalocyanines), chlorins (e.g. tin chlorin e6, poly-lysine derivatives of tin chlorin e6, m-tetrahydroxyphenyl chlorin, benzoporphyrin derivatives, tin etiopurpurin), bacteriochlorins, phenothiazines (e.g. toluidine blue 0, methylene blue, dimethylmethylene blue), phenazines (e.g. neutral red), acridines (e.g. acriflavine, proflavin, acridine orange, aminacrine), texaphyrins, cyanines (e.g. merocyanine 540), anthracycline (e.g. adriamycin and epirubicin), pheophorbides, sapphyrins, fullerene, halogenated xanthenes (e.g. rose bengal), perylenequinonoid pigments (e.g. hypericin, hypocrellin), gilvocarcins, terthiophenes, benzophenanthridines, psoralens and riboflavin. Other possibilities are arianor steel blue, tryptan blue, crystal violet, azure blue cert, azure B chloride, azure 2, azure A chloride, azure B tetrafluoroborate, thionin, azure A eosinate, azure B eosinate, azure mix sicc. and azure II eosinate.

Exemplary xanthene dyes include, but are not limited to, Eosin B (4′,5′-dibromo,2′,7′-dinitro-fluorescein, dianion); eosin Y; eosin Y (2′,4′,5′,7′-tetrabromo-fluorescein, dianion); eosin (2′,4′,5′,7′-tetrabromo-fluorescein, dianion); eosin (2′,4′,5′,7′-tetrabromo-fluorescein, dianion) methyl ester; eosin (2′,4′,5′,7′-tetrabromo-fluorescein, monoanion)p-isopropylbenzyl ester; eosin derivative (2′,7′-dibromo-fluorescein, dianion); eosin derivative (4′,5′-dibromo-fluorescein, dianion); eosin derivative (2′,7′-dichloro-fluorescein, dianion); eosin derivative (4′,5′-dichloro-fluorescein, dianion); eosin derivative (2′,7′-diiodo-fluorescein, dianion); eosin derivative (4′,5′-diiodo-fluorescein, dianion); eosin derivative (tribromo-fluorescein, dianion); eosin derivative (2′,4′,5′,7′-tetrachloro-fluorescein, dianion); eosin; eosin dicetylpyridinium chloride ion pair; erythrosin B (2′,4′,5′,7′-tetraiodo-fluorescein, dianion); erythrosin; erythrosin dianion; erythrosin B; fluorescein; fluorescein dianion; phioxin B (2′,4′,5′,7′-tetrabromo-3,4,5,6-tetrachloro-fluorescein, dianion); phloxin B (tetrachloro-tetrabromo-fluorescein); phloxine B; rose bengal (3,4,5,6-tetrachloro-2′,4′,5′,7′-tetraiodofluorescein, dianion); rose bengal; rose bengal dianion; rose bengal O-methyl-methylester; rose bengal 6′-O-acetyl ethyl ester; rose bengal benzyl ester diphenyl-diiodonium salt; rose bengal benzyl ester triethylammonium salt; rose bengal benzyl ester, 2,4,6,-triphenylpyrilium salt; rose bengal benzyl ester, benzyltriphenyl-phosphonium salt; rose bengal benzyl ester, benzyltriphenyl phosphonium salt; rose bengal benzyl ester, diphenyl-iodonium salt; rose bengal benzyl ester, diphenyl-methylsulfonium salt; rose bengal benzyl ester, diphenyl-methyl-sulfonium salt; rose bengal benzyl ester, triethyl-ammonium salt; rose bengal benzyl ester, triphenyl pyrilium; rose bengal bis(triethyl-ammonium) salt) (3,4,5,6-tetrachloro-2′,4′,5′,7′-tetraiodofluorescein, bis(triethyl-ammonium salt); rose bengal bis (triethyl-ammonium) salt; rose bengal bis(benzyl-triphenyl-phosphonium) salt (3,4,5, 6-tetrachloro-2′,4′,5′,7′-tetraiodofluorescein, bis(benzyl-triphenyl-phosphonium) salt); rose bengal bis(diphenyl-iodonium) salt (3,4,5,6-tetrachloro-2′,4′,5′,7′-tetraiodofluorescein, bis(diphenyl-iodonium) salt); rose bengal di-cetyl-pyridinium chloride ion pair; rose bengal ethyl ester triethyl ammonium salt; rose bengal ethyl ester triethyl ammonium salt; rose bengal ethyl ester; rose bengal methyl ester; rose bengal octyl ester tri-n-butyl-ammonium salt RB; rose bengal, 6′-O-acetyl-, and ethyl ester.

Exemplary methylene blue derivatives include, but are not limited to, 1-methyl methylene blue; 1,9-dimethyl methylene blue; methylene blue; methylene blue (16 μM); methylene blue (14 μM); methylene violet; bromomethylene violet; 4-iodomethylene violet; 1,9-dimethyl-3-dimethyl-amino-7-diethyl-amino-phenothiazine; and 1,9-dimethyl-3-diethylamino-7-dibutyl-amino-phenothiazine.

Exemplary naphthalimides blue derivatives include, but are not limited to, N,N′-bis-(hydroperoxy-2-methoxyethyl)-1,4,5,8-naphthaldiimide; N-(hydroperoxy-2-methoxyethyl)-1,8-naphthalimide; 1, 8-naphthalimide; N,N′-bis(2,2-dimethoxyethyl)-1,4,5,8-naphthaldiimide; and N,N′-bis(2,2-dimethylpropyl)-1,4,5, 8-naphthaldiimide.

Exemplary chlorophylls dyes include, but are not limited to, chlorophyll a; chlorophyll b; oil soluble chlorophyll; bacteriochlorophyll a; bacteriochlorophyll b; bacteriochlorophyll c; bacteriochlorophyll d; protochlorophyll; protochlorophyll a; amphiphilic chlorophyll derivative 1; and amphiphilic chlorophyll derivative 2.

Exemplary coumarins include, but are not limited to, 3-benzoyl-7-methoxycoumarin; 7-diethylamino-3-thenoylcoumarin; 5,7-dimethoxy-3-(1-naphthoyl) coumarin; 6-methylcoumarin; 2H-selenolo[3,2-g] [1] benzopyran-2-one; 2H-selenolo[3,2-g] [1] benzothiopyran-2-one; 7H-selenolo[3,2-g] [1] benzoseleno-pyran-7-one; 7H-selenopyrano[3,2-f] [1] benzofuran-7-one; 7H-selenopyrano[3,2-f] [1] benzo-thiophene-7-one; 2H-thienol[3,2-g] [1] benzopyran-2-one; 7H-thienol[3,2-g] [1] benzothiopyran-7-one; 7H-thiopyrano[3,2-f] [1] benzofuran-7-one; coal tar mixture; khellin; RG 708; RG277; and visnagin.

Exemplary cyanines include, but are not limited to, benzoselenazole dye; benzoxazole dye; 1,1′-diethyloxacarbocyanine; 1,1′-diethyloxadicarbocyanine; 1,1′-diethylthiacarbocyanine; 3,3′-dialkylthiacarbocyanines (n=2-18); 3,3 diethylthiacarbocyanine iodide; 3,3′-dihexylselenacarbocyanine; kryptocyanine; MC540 benzoxazole derivative; MC540 quinoline derivative; merocyanine 540; and meso-ethyl, 3,3′-dihexylselenacarbocyanine.

Exemplary porphyrins include, but are not limited to, 5-azaprotoporphyrin dimethylester; bis-porphyrin; coproporphyrin III; coproporphyrin III tetramethylester; deuteroporphyrin; deuteroporphyrin IX dimethylester; diformyldeuteroporphyrin IX dimethylester; dodecaphenylporphyrin; hematoporphyrin; hematoporphyrin IX; hematoporphyrin monomer; hematoporphyrin dimer; hematoporphyrin derivative; hematoporphyrin IX dihydrochloride; hematoporphyrin dihydrochloride; hematoporphyrin IX dimethylester; haematoporphyrin IX dimethylester; mesoporphyrin dimethylester; mesoporphyrin IX dimethylester; monoformyl-monovinyl-deuteroporphyrin IX dimethylester; monohydroxyethylvinyl deuteroporphyrin; 5,10,15,20-tetra(o-hydroxyphenyl) porphyrin; 5,10,15,20-tetra(m-hydroxyphenyl) porphyrin; 5,10,15,20-tetrakis-(m-hydroxyphenyl) porphyrin; 5,10,15,20-tetrakis-hydroxyphenyl) porphyrin; 5,10,15,20-tetrakis(3-methoxyphenyl) porphyrin; 5, 10,15,20-tetrakis (3,4-dimethoxyphenyl) porphyrin; 5, 10,15,20-tetrakis (3,5-dimethoxyphenyl) porphyrin; 5, 10,15,20-tetrakis (3,4,5-trimethoxyphenyl) porphyrin; 2,3,7,8,12,13,17,18-octaethyl-5,10,15,20-tetraphenylporphyrin; Photofrin®; Photofrin® II; porphyrin C; protoporphyrin; protoporphyrin IX; protoporphyrin dimethylester; protoporphyrin IX dimethylester; protoporphyrin propylaminoethylformamide iodide; protoporphyrin N,N-dimethylaminopropylformamide; protoporphyrin propylaminopropylformamide iodide; protoporphyrin butylformamide; protoporphyrin N,N-dimethylamino-formamide; protoporphyrin formamide; sapphyrin 13,12,13,22-tetraethyl-2,7,18,23 tetramethyl sapphyrin-8,17-dipropanol; sapphyrin 2 3,12,13,22-tetraethyl-2,7,18,23 tetramethyl sapphyrin-8-monoglycoside; sapphyrin 3; meso-tetra-(4-N-carboxyphenyl)-porphine; tetra-(3-methoxyphenyl)-porphine; tetra-(3-methoxy-2,4-difluorophenyl)-porphine; 5,10,15,20-tetrakis(4-N-methylpyridyl) porphine; meso-tetra-(4-N-methylpyridyl)-porphine tetrachloride; meso-tetra(4-N-methylpyridyl)-porphine; meso-tetra-(3-N-methylpyridyl)-porphine; meso-tetra-(2-N-methylpyridyl)-porphine; tetra(4-N,N,N-trimethylanilinium) porphine; meso-tetra-(4-N,N,N″-trimethylamino-phenyl) porphine tetrachloride; tetranaphthaloporphyrin; 5,10,15,20-tetraphenylporphyrin; tetraphenylporphyrin; meso-tetra-(4-N-sulfonatophenyl)-porphine; tetraphenylporphine tetrasulfonate; meso-tetra(4-sulfonatophenyl)porphine; tetra(4-sulfonatophenyl)porphine; tetraphenylporphyrin sulfonate; meso-tetra(4-sulfonatophenyl)porphine; tetrakis(4-sulfonatophenyl)porphyrin; meso-tetra(4-sulfonatophenyl)porphine; meso(4-sulfonatophenyl)porphine; meso-tetra(4-sulfonatophenyl)porphine; tetrakis(4-sulfonatophenyl)porphyrin; meso-tetra(4-N-trimethylanilinium)-porphine; uroporphyrin; uroporphyrin I; and uroporphyrin IX.

Exemplary metalloporphyrins include, but are not limited to, cadmium (II) chlorotexaphyrin nitrate; cadmium (II) meso-diphenyl tetrabenzoporphyrin; cadmium meso-tetra-(4-N-methylpyridyl)-porphine; cadmium (II) texaphyrin; cadmium (II) texaphyrin nitrate; cobalt meso-tetra-(4-N-methylpyridyl)-porphine; cobalt (II) meso(4-sulfonatophenyl)-porphine; copper hematoporphyrin; copper meso-tetra-(4-N-methylpyridyl)-porphine; copper (II) meso(4-sulfonatophenyl)-porphine; Europium (III) dimethyltexaphyrin dihydroxide; gallium tetraphenylporphyrin; iron meso-tetra(4-N-methylpyridyl)-porphine; lutetium (III) tetra(N-methyl-3-pyridyl)-porphyrin chloride; magnesium (II) meso-diphenyl tetrabenzoporphyrin; magnesium tetrabenzoporphyrin; magnesium tetraphenylporphyrin; magnesium (II) meso(4-sulfonatophenyl)-porphine; magnesium (II) texaphyrin hydroxide metalloporphyrin; magnesium meso-tetra-(4-N-methylpyridyl)-porphine; manganese meso-tetra-(4-N-methylpyridyl)-porphine; nickel meso-tetra(4-N-methylpyridyl)-porphine; nickel (II) meso-tetra(4-sulfonatophenyl)-porphine; palladium (II) meso-tetra-(4-N-methylpyridyl)-porphine; palladium meso-tetra-(4-N-methylpyridyl)-porphine; palladium tetraphenylporphyrin; palladium (II) meso(4-sulfonatophenyl)-porphine; platinum (II) meso(4-sulfonatophenyl)-porphine; samarium (II) dimethyltexaphyrin dihydroxide; silver (II) meso(4-sulfonatophenyl)-porphine; tin (IV) protoporphyrin; tin meso-tetra-(4-N-methylpyridyl)-porphine; tin meso-tetra(4-sulfonatophenyl)-porphine; tin (IV) tetrakis(4-sulfonatophenyl) porphyrin dichloride; zinc (II) 15-aza-3,7,12,18-tetramethyl-porphyrinato-13,17-diyl-dipropionic acid-dimethylester; zinc (II) chlorotexaphyrin chloride; zinc coproporphyrin III; zinc (II) 2,11,20,30-tetra-(1,1-dimethyl-ethyl)tetranaphtho(2,3-b:2′,3′-g:2′3′-1:-2′3′″-q)porphyrazine; zinc (II) 2-(3-pyridyloxy)benzo[b]-10,19,28-tri(1,1-dimethylethyl)trinaphtho[2′,3′-g:2″3″1::2′″,3′″-q]porphyrazine; zinc (II) 2,18-bis-(3-pyridyloxy)dibenzo[b,1]-10,26-di(1,1-dimethyl-ethyl)dinaphtho-[2′,3′-g:2′″,3′″-q]porphyrazine; zinc (II) 2,9-bis-(3-pyridyloxy)dibenzo[b,g]-17,26-di(1,1-dimethyl-ethyl)dinaphtho[2″,3″-1:2′″,3′″q]porphyrazine; zinc (II) 2,9,16-tris-(3-pyridyloxy) tribenzo[b,g,1]-24=(1,1-dimethyl-ethyl)naphtho[2′″,3′-q]porphyrazine; zinc (II) 2,3-bis-(3-pyridyloxy)benzo[b]-10,19,28-tri(1,1-dimethyl-ethyl)trinaphtho[2′,3′-g:2″,3″1:2′″3′″-q]porphyrazine; zinc (II) 2,3,18,19-tetrakis-(3-pyridyloxy) dibenzo[b,1]-10,26-di(1,1-dimethyl-ethyl)trinaphtho[2′,3′-g:2′″,3′″-q]porphyrazine; zinc (II) 2,3,9,10-tetrakis-(3-pyridyloxy) dibenzo[b,g]-17,26-di(1,1-dimethyl-ethyl)dinaphtho[2″,3′-1:2′″,3′″-q]porphyrazine; zinc (II) 2,3,9,10,16,17-hexakis-(3-pyridyloxy)tribenzo[b,g,1]-24-(1,1-dimethyl-ethyl)naphtho[2′″,3′″-q]porphyrazine; zinc (II) 2-(3-N-methyl)pyridyloxy)benzo[b]-10,19,28-tri(1,1-dimethyl-ethyl)trinaphtho[2′,3′-g:2″,3″1:2′″,3′″-q]porphyrazine monoiodide; zinc (II) 2,18-bis-(3-(N-methyl)pyridyloxy)dibenzo[b,1]-10,26-di(1,1-dimethylethyl)dinaphtho[2′,3′-g 2′″,3′″-q]porphyrazine diiodide; zinc (II) 2,9-bis-(3-(N-methyl)pyridyloxy)dibenzo[b,g]-17,26-di(1,1-dimethylethyl)dinaphtho[2″,3″-1:2′″,3′″-q]porphyrazine diiodide; zinc (II) 2,9,16-tris-(3-(N-methyl-pyridyloxy)tribenzo[b,g,1]-24-(1,1-dimethylethyl-) naphtho[2′″,3′″-q]porphyrazine triiodide; zinc (II) 2,3-bis-(3-(N-methyl)pyridyloxy)benzo[b]-10,19,28-tri(1,1-dimethylethyl)trinaphtho[2′,3′-g:2″,3″-1:2′″,3′″-q]porphyrazine diiodide; zinc (II) 2,3,18,19-tetrakis-(3-(N-methyl)pyridyloxy)dibenzo[b,1]-10,26-di(1,1-dimethyl)dinaphtho[2′,3′-g:2′″,3′″-q]porphyrazine tetraiodide; zinc (II) 2,3,9,10-tetrakis-(3-(N-methyl)pyridyloxy)dibenzo[g,g]-17,26-di(1,1-dimethylethyl)dinaphtho[2″,3″-1:2′″,3′″-q]porphyrazine tetraiodide; zinc (II) 2,3,9,10,16,17-hexakis-(3-(N-methyl)pyridyloxy)tribenzo[b,g,1]-24-(1-,1-dimethylethyl)naphtho[2″′,3′″q]porphyrazine hexaiodide; zinc (II) meso-diphenyl tetrabenzoporphyrin; zinc (II) meso-triphenyl tetrabenzoporphyrin; zinc (II) meso-etrakis(2,6-dichloro-3-sulfonatophenyl) porphyrin; zinc (II) meso-tetra-(4-N-methylpyridyl)-porphine; zinc (II) 5,10,15,20-meso-tetra(4-octyl-phenylpropynyl)-porphine; zinc porphyrin C; zinc protoporphyrin; zinc protoporphyrin IX; zinc (II) meso-triphenyl-tetrabenzoporphyrin; zinc tetrabenzoporphyrin; zinc (II) tetrabenzoporphyrin; zinc tetranaphthaloporphyrin; zinc tetraphenylporphyrin; zinc (II) 5,10,15,20-tetraphenylporphyrin; zinc (II) meso (4-sulfonatophenyl)-porphine; and zinc (II) texaphyrin chloride.

Exemplary metallophthalocyanines include, but are not limited to, aluminum mono-(6-carboxy-pentyl-amino-sulfonyl)-trisulfo-phthalocyanine; aluminum di-(6-carboxy-pentyl-amino-sulfonyl)-trisulfophthalocyanine; aluminum (III) octa-n-butoxy phthalocyanine; aluminum phthalocyanine; aluminum (III) phthalocyanine disulfonate; aluminum phthalocyanine disulfonate; aluminum phthalocyanine disulfonate (cis isomer); aluminum phthalocyanine disulfonate (clinical prep.); aluminum phthalocyanine phthalimido-methyl sulfonate; aluminum phthalocyanine sulfonate; aluminum phthalocyanine trisulfonate; aluminum (III) phthalocyanine trisulfonate; aluminum (III) phthalocyanine tetrasulfonate; aluminum phthalocyanine tetrasulfonate; chloroaluminum phthalocyanine; chloroaluminum phthalocyanine sulfonate; chloroaluminum phthalocyanine disulfonate; chloroaluminum phthalocyanine tetrasulfonate; chloroaluminum-t-butyl-phthalocyanine; cobalt phthalocyanine sulfonate; copper phthalocyanine sulfonate; copper (II) tetra-carboxy-phthalocyanine; copper (II)-phthalocyanine; copper t-butyl-phthalocyanine; copper phthalocyanine sulfonate; copper (II) tetrakis-[methylene-thio[(dimethyl-amino)methylidyne]]phthalocyanine tetrachloride; dichlorosilicon phthalocyanine; gallium (III) octa-n-butoxy phthalocyanine; gallium (II) phthalocyanine disulfonate; gallium phthalocyanine disulfonate; gallium phthalocyanine tetrasulfonate-chloride; gallium (II) phthalocyanine tetrasulfonate; gallium phthalocyanine trisulfonate-chloride; gallium (II) phthalocyanine trisulfonate; GaPcS₁tBu₃; GaPcS₂tBu₂; GaPcS₃tBu₁; germanium (IV) octa-n-butoxy phthalocyanine; germanium phthalocyanine derivative; silicon phthalocyanine derivative; germanium (IV) phthalocyanine octakis-alkoxy-derivatives; iron phthalocyanine sulfonate; lead (II) 2,3,9,10,16,17,23,24-octakis(3,6-dioxaheptyloxy) phthalocyanine; magnesium t-butyl-phthalocyanine; nickel (II) 2,3,9,10,16,17,23,24-octakis(3,6-dioxaheptyloxy) phthalocyanine; palladium (II) octa-n-butoxy phthalocyanine; palladiun (II) tetra(t-butyl)-phthalocyanine; (diol) (t-butyl)₃-phthalocyanato palladium(II); ruthenium(II) dipotassiumbis(triphenyl-phosphine-monosulphonate) phthalocyanine; silicon phthalocyanine bis(tri-n-hexyl-siloxy)-; silicon phthalocyanine bis(tri-phenyl-siloxy); HOSiPcOSi(CH₃)₂(CH₂)₃N(CH₃)₂; HOSiPcOSi(CH₃)₂(CH₂)₃N(CH₂CH₃)₂; SiPc[OSi(CH₃)₂(CH₂)₃N(CH₃)₂]₂; SiPc[OSi(CH₃)₂(CH₂)₃N(CH₂CH₃)—(CH₂)₂N(CH₃)₂]₂; tin (IV) octa-n-butoxy phthalocyanine; vanadium phthalocyanine sulfonate; zinc (II) octa-n-butoxy phthalocyanine; zinc (II) 2,3,9,10,16,17,23,24-octakis(2-ethoxy-ethoxy) phthalocyanine; zinc (II) 2,3,9,10,16,17,23,24-octakis(3,6-dioxaheptyloxy) phthalocyanine; zinc (II) 1,4,8,11,15,18,22,25-octa-n-butoxy-phthalocyanine; zn(II)-phthalocyanine-octabutoxy, zn(II)-phthalocyanine; zinc phthalocyanine; zinc (II) phthalocyanine; zinc phthalocyanine and perdeuterated zinc phthalocyanine; zinc (II) phthalocyanine disulfonate; zinc phthalocyanine disulfonate; zinc phthalocyanine sulfonate; zinc phthalocyanine tetrabromo-; zinc (II) phthalocyanine tetra-t-butyl-; zinc (II) phthalocyanine tetra-(t-butyl)-; zinc phthalocyanine tetracarboxy-; zinc phthalocyanine tetrachloro-; zinc phthalocyanine tetrahydroxyl; zinc phthalocyanine tetraiodo-; zinc ((I) tetrakis-(1,1-dimethyl-2-phthalimido)ethyl phthalocyanine; zinc (II) tetrakis-(1,1-dimethyl-2-amino)-ethyl-phthalocyanine; zinc (II) phthalocyanine tetrakis(1,1-dimethyl-2-trimethyl ammonium)ethyl tetraiodide; zinc phthalocyanine tetrasulphonate; zinc phthalocyanine tetrasulfonate; zinc (II) phthalocyanine tetrasulfonate; zinc (II) phthalocyanine trisulfonate; zinc phthalocyanine trisulfonate; zinc (II) (t-butyl)₃-phthalocyanine diol; zinc tetradibenzobarreleno-octabutoxy-phthalocyanine; zinc (II) 2,9,16,23,-tetrakis-(3-(N-methyl)pyridyloxy)phthalocyanine tetraiodide; and zinc (II) 2,3,9,10,16,17,23,24-octakis-(3-(N-methyl)pyridyloxy)phthalocyanine complex octaiodide; and zinc (II) 2,3,9,10,16,17,23,24-octakis-(3-pyridyloxy)phthalocyanine.

Exemplary naphthalocyanines include, but are not limited to, aluminum t-butyl-chloronaphthalocyanine; silicon bis(dimethyloctadecylsiloxy) 2,3-naphthalocyanine; silicon bis(dimethyloctadecylsiloxy) naphthalocyanine; silicon bis(dimethylthexylsiloxy) 2,3-naphthalocyanine; silicon bis(dimethylthexylsiloxy) naphthalocyanine; silicon bis(t-butyldimethylsiloxy) 2,3-naphthalocyanine; silicon bis(tert-butyldimethylsiloxy) naphthalocyanine; silicon bis(tri-n-hexylsiloxy) 2,3-naphthalocyanine; silicon bis(tri-n-hexylsiloxy) naphthalocyanine; silicon naphthalocyanine; t-butylnaphthalocyanine; zinc (II) naphthalocyanine; zinc (II) tetraacetyl-amidonaphthalocyanine; zinc (II) tetraaminonaphthalocyanine; zinc (II) tetrabenzamidonaphthalocyanine; zinc (II) tetrahexylamidonaphthalocyanine; zinc (II) tetramethoxy-benzamidonaphthalocyanine; zinc (II) tetramethoxynaphthalocyanine; zinc naphthalocyanine tetrasulfonate; and zinc (II) tetradodecylamidonaphthalocyanine.

Exemplary perylenequinones include, but are not limited to, hypericins such as hypericin; hypericin monobasic sodium salt; di-aluminum hypericin; di-copper hypericin; gadolinium hypericin; terbium hypericin, hypocrellins such as acetoxy hypocrellin A; acetoxy hypocrellin B; acetoxy iso-hypocrellin A; acetoxy iso-hypocrellin B; 3,10-bis[2-(2-aminoethylamino)ethanol]hypocrellin B; 3,10-bis[2-(2-aminoethoxy)ethanol]hypocrellin B; 3,10-bis[4-(2-aminoethyl)morpholine]hypocrellin B; n-butylaminated hypocrellin B; 3,10-bis(butylamine) hypocrellin B; 4,9-bis(butylamine) hypocrellin B; carboxylic acid hypocrellin B; cystamine-hypocrellin B; 5-chloro hypocrellin A or 8-chloro hypocrellin A; 5-chloro hypocrellin B or 8-chloro hypocrellin B; 8-chloro hypocrellin B; 8-chloro hypocrellin A or 5-chloro hypocrellin A; 8-chloro hypocrellin B or 5-chloro hypocrellin B; deacetylated aldehyde hypocrellin B; deacetylated hypocrellin B; deacetylated hypocrellin A; deacylated, aldehyde hypocrellin B; demethylated hypocrellin B; 5,8-dibromo hypocrellin A; 5,8-dibromo hypocrellin B; 5,8-dibromo iso-hypocrellin B; 5,8-dibromo[1,12-CBr═CMeCBr(COMe)] hypocrellin B; 5,8-dibromo[1,12-CHBrC═CH₂)CBr(COMe)] hypocrellin B; 5,8-dibromo[1-CH₂COMe, 12-COCOCH₂Br-]hypocrellin B; 5,8-dichloro hypocrellin A; 5,8-dichloro hypocrellin B; 5,8-dichlorodeacytylated hypocrellin B; 5,8-diiodo hypocrellin A; 5,8-diiodo hypocrellin B; 5,8-diiodo[1,12-CH═CMeCH(COCH₂I₂)-] hypocrellin B; 5,8-diiodo[1,12-CH₂C(CH₂I)═C(COMe)-] hypocrellin B; 2-(N,N-diethylamino) ethylaminated hypocrellin B; 3,10-bis[2-(N,N-diethylamino)-ethylamine]hypocrellin B; 4,9-bis[2-(N,N-diethyl-amino)-ethylamine] iso-hypocrellin B; dihydro-1,4-thiazine carboxylic acid hypocrellin B; dihydro-1,4-thiazine hypocrellin B; 2-(N,N-dimethylamino) propylamine hypocrellin B; dimethyl-1,3,5,8,10,12-hexamethoxy-4,9-perylenequinone-6,7-di acetate; dimethyl-5,8-dihydroxy-1,3, 10,13-tetramethoxy-4,9-perylenequinone-6,7-diacetate; 2,11-dione hypocrellin A; ethanolamine hypocrellin B; ethanolamine iso-hypocrellin B; ethylenediamine hypocrellin B; 1-hydroxy hypocrellin B or 2-hydroxy hypocrellin B; hypocrellin A; hypocrellin B; 5-iodo[1,12-CH₂C(CH₂₁)═C(COMe)]hypocrellin B; 8-iodo[1,12-CH₂C(CH₂₁)═C(COMe)-] hypocrellin B; 9-methylamino iso-hypocrellin B; 3,10-bis[2-(N,N-methylamino)propylamine]hypocrellin B; 4,9-bis(methylamine iso-hypocrellin B; 14-methylamine iso-hypocrellin B; 4-methylamine iso-hypocrellin B; methoxy hypocrellin A; methoxy hypocrellin B; methoxy iso-hypocrellin A; methoxy iso-hypocrellin B; methylamine hypocrellin B; 2-morpholino ethylaminated hypocrellin B; pentaacetoxy hypocrellin A; PQP derivative; tetraacetoxy hypocrellin B; 5,8,15-tribromo hypocrellin B; calphostin C, Cercosporins such as acetoxy cercosporin; acetoxy iso-cercosporin; aminocercosporin; cercosporin; cercosporin+iso-cercosporin (1/1 molar); diaminocercosporin; dimethylcercosporin; 5,8-dithiophenol cercosporin; iso-cercosporin; methoxycercosporin; methoxy iso-cercosporin; methylcercosporin; noranhydrocercosporin; elsinochrome A; elsinochrome B; phleichrome; and rubellin A.

Exemplary pheophorbides include, but are not limited to, pheophorbide A; methyl 13¹-deoxy-20-formyl-7,8-vic-dihydro-bacterio-meso-pheophorbide A; methyl-2-(1-dodecyloxyethyl)-2-devinyl-pyropheophorbide A; methyl-2-(1-heptyl-oxyethyl)-2-devinyl-pyropheophorbide A; methyl-2-(1-hexyl-oxyethyl)-2-devinyl-pyropheophorbide A; methyl-2-(1-methoxy-ethyl)-2-devinyl-pyropheophorbide A; methyl-2-(1-pentyl-oxyethyl)-2-devinyl-pyropheophorbide A; magnesium methyl bacteriopheophorbide D; methyl-bacteriopheophorbide D; and pheophorbide.

Exemplary pheophytins include, but are not limited to, bacteriopheophytin A; bacteriopheophytin B; bacteriopheophytin C; bacteriopheophytin D; 10-hydroxy pheophytin a; pheophytin; pheophytin A; and protopheophytin.

Exemplary phthalocyanines include, but are not limited to, (diol) (t-butyl)₃-phthalocyanine; (t-butyl)₄-phthalocyanine; cis-octabutoxy-dibenzo-dinaphtho-porphyrazine; trans-octabutoxy-dibenzo-dinaphtho-porphyrazine; 2,3,9,10,16,17,23,24-octakis2-ethoxyethoxy)phthalocyanine; 2,3,9,10,16,17,23,24-octakis(3,6-dioxaheptyloxy) phthalocyanine; octa-n-butoxy phthalocyanine; phthalocyanine; phthalocyanine sulfonate; phthalocyanine tetrasulphonate; phthalocyanine tetrasulfonate; t-butyl-phthalocyanine; tetra-t-butyl phthalocyanine; and tetradibenzobarreleno-octabutoxy-phthalocyanine.

Exemplary porphycenes include, but are not limited to, 2,3-(2₃-carboxy-2₄-methoxycarbonyl benzo)-7,12,17-tris(2-methoxyethyl) porphycene; 2-(2-hydroxyethyl)-7,12,17-tri(2-methoxyethyl) porphycene; 2-(2-hydroxyethyl)-7,12,17-tri-n-propyl-porphycene; 2-(2-methoxyethyl)-7,12,17-tri-n-propyl-porphycene; 2,7,12,17-tetrakis(2-methoxyethyl) porphycene; 2,7,12,17-tetrakis(2-methoxyethyl)-9-hydroxy-porphycene; 2,7,12,17-tetrakis(2-methoxyethyl)-9-methoxy-porphycene; 2,7,12,17-tetrakis(2-methoxyethyl)-9-n-hexyloxy-porphycene; 2,7,12,17-tetrakis(2-methoxyethyl)-9-acetoxy-porphycene; 2,7,12,17-tetrakis(2-methoxyethyl)-9-caproyloxy-porphycene; 2,7,12,17-tetrakis(2-methoxyethyl)-9-pelargonyloxy-porphycene; 2,7,12,17-tetrakis(2-methoxyethyl)-9-stearoyloxy-porphycene; 2,7,12,17-tetrakis(2-methoxyethyl)-9-(N-t-butoxycarbonylglycinoxy) porphycene; 2,7,12,17-tetrakis(2-methoxyethyl)-9-[4-(.beta.-apo-7-carotenyl)benzoylox-yl-porphycene; 2,7,12,17-tetrakis(2-methoxyethyl)-9-amino-porphycene; 2,7,12,17-tetrakis(2-methoxyethyl)-9-acetamido-porphycene; 2,7,12,17-tetrakis(2-methoxyethyl)-9-glutaramido-porphycene; 2,7,12,17-terakis(2-methoxyethyl)-9-(methyl-glutaramido)-porphycene; 2,7,12,17-tetrakis(2-methoxyethyl)-9-(glutarimido)-porphycene; 2,7,12,17-tetrakis(2-methoxyethyl)-3-(N,N-dimethylaminomethyl)-porphycene; 2,7,12,17-tetrakis(2-methoxyethyl)-3-(N,N-dimethylaminomethyl)-porphycene hydrochloride; 2,7,12,17-tetrakis(2-ethoxyethyl)-porphycene; 2,7,12,17-tetra-n-propyl-porphycene; 2,7,12,17-tetra-n-propyl-9-hydroxy-porphycene; 2,7,12,17-tetra-n-propyl-9-methoxy-porphycene; 2,7,12,17-tetra-n-propyl-9-acetoxy porphycene; 2,7,12,17-tetra-n-propyl-9-(t-butyl glutaroxy)-porphycene; 2,7,12,17-tetra-n-propyl-9-(N-t-butoxycarbonylglycinoxy)-porphycene; 2,7,12,17-tetra-n-propyl-9-(4-N-t-butoxy-carbonyl-butyroxy)-porphycene, 2,7,12,17-tetra-n-propyl-9-amino-porphycene; 2,7,12,17-tetra-n-propyl-9-acetamido-porphycene; 2,7,12,17-tetra-n-propyl-9-glutaramido-porphycene; 2,7,12,17-tetra-n-propyl-9-(methyl glutaramido)-porphycene; 2,7,12,17-tetra-n-propyl-3-(N,N-diethylarninomethyl) porphycene; 2,7,12,17-tetra-n-propyl-9,10-benzo porphycene; 2,7,12,17-tetra-n-propyl-9-p-benzoyl carboxy-porphycene; 2,7,12,17-tetra-n-propyl-porphycene; 2,7,12,17-tetra-t-butyl-3,6;13,16-dibenzo-porphycene; 2,7-bis(2-hydroxyethyl)-12,17-di-n-propyl-porphycene; 2,7-bis(2-methoxyethyl)-12,17-di-n-propyl-porphycene; and porphycene.

Exemplary psoralens include, but are not limited to, psoralen; 5-methoxypsoralen; 8-methoxypsoralen; 5,8-dimethoxypsoralen; 3-carbethoxypsoralen; 3-carbethoxy-pseudopsoralen; 8-hydroxypsoralen; pseudopsoralen; 4,5′,8-trimethylpsoralen; allopsoralen; 3-aceto-allopsoralen; 4,7-dimethyl-allopsoralen; 4,7,4′-trimethyl-allopsoralen; 4,7,5′-trimethyl-allopsoralen; isopseudopsoralen; 3-acetoisopseudopsoralen; 4,5′-dimethyl-isopseudopsoralen; 5′,7-dimethyl-isopseudopsoralen; pseudoisopsoralen; 3-acetopseudoisopsoralen; 3/4′,5′-trimethyl-aza-psoralen; 4,4′,8-trimethyl-5 anino-methylpsoralen; 4,4′,8-trimethyl-phthalamyl-psoralen; 4,5′,8-trimethyl-4′-aminomethyl psoralen; 4,5′,8-trimethyl-bromopsoralen; 5-nitro-8-methoxy-psoralen; 5′-acetyl-4, 8-dimethyl-psoralen; 5′-aceto-8-methyl-psoralen; and 5′-aceto-4,8-dimethyl-psoralen. Exemplary purpurins include octaethylpurpurin; octaethylpurpurin zinc; oxidized octaethylpurpurin; reduced octaethylpurpurin; reduced octaethylpurpurin tin; purpurin 18; purpurin-18; purpurin-18-methyl ester; purpurin; tin ethyl etiopurpurin I; Zn(II) aetio-purpurin ethyl ester; and zinc etiopurpurin.

Exemplary quinones include, but are not limited to, 1-amino-4,5-dimethoxy anthraquinone; 1,5-diamino-4,8-dimethoxy anthraquinone; 1,8-diamino-4,5-dimethoxy anthraquinone; 2,5-diamino-1,8-dihydroxy anthraquinone; 2,7-diamino-1, 8-dihydroxy anthraquinone; 4,5-diamino-1, 8-dihydroxy anthraquinone; mono-methylated 4,5- or 2,7-diamino-1,8-dihydroxy anthraquinone; anthralin (keto form); anthralin; anthralin anion; 1,8-dihydroxy anthraquinone; 1,8-dihydroxy anthraquinone (Chrysazin); 1,2-dihydroxy anthraquinone; 1,2-dihydroxy anthraquinone (Alizarin); 1,4-dihydroxy anthraquinone (Quinizarin); 2,6-dihydroxy anthraquinone; 2,6-dihydroxy anthraquinone (Anthraflavin); 1-hydroxy anthraquinone (Erythroxy-anthraquinone); 2-hydroxy-anthraquinone; 1,2,5,8-tetra-hydroxy anthraquinone (Quinalizarin); 3-methyl-1,6, 8-trihydroxy anthraquinone (Emodin); anthraquinone; anthraquinone-2-sulfonic acid; benzoquinone; tetramethyl benzoquinone; hydroquinone; chlorohydroquinone; resorcinol; and 4-chlororesorcinol.

In some embodiments, exemplary PSs include, without limitation, thiazine dyes such as phenothiazine dyes (e.g., methylene blue, dimethyl methylene blue, new methylene blue N, neutral red, toluidine blue O, thionine, azure C), acridine dyes (e.g., acridine orange, acridine yellow, proflavin.), coumarin dyes (e.g., thiocoumarin), xanthene dyes (e.g., eosin, fluorescein, rose bengal), phenazines (e.g., neutral red), phenoxaziniums (e.g., brilliant cresyl blue), fluorene derivatives (e.g., fluorine, fluorenones), psoralens, naphthalocyanines, porphyrin and benzoporphyin derivatives (e.g., copper porphyrin, zinc tetraphenylporphyrin tetrasulfonate, and chlorins such as 5,10,15,20-tetrakis(m-hydroxyphenyl)chlorine), phthalocyanines (e.g., pthalocyaninetetrasulfonic acid as well as zinc-, aluminium- or silicon-phthalocyanines, which may be sulfonated, including aluminium phthalocyanine monosulfonates (AlPcS), aluminium phthalocyanine disulfonates (AlPcS2), aluminium phthalocyanine trisulfonates (AlPcS3) or aluminium phthalocyanine tetrasulfonates (AlPcS4)), as well as mixtures thereof.

In some embodiments, the PS is a xanthenes dye as described herein.

In some embodiments, the xanthene photosensitizer is one or more of Rose Bengal (free acid/free alcohol); Rose Bengal salts such as Rose Bengal dilithium salt, Rose Bengal disodium salt (CAS 632-69-9), Rose Bengal dipotassium salt, Rose Bengal diammonium salt, and Rose Bengal di-(substituted ammonium) salt (e.g., bis(triethylammonium, CAS 91491-51-9) and bis[tri-(n-butyl) ammonium); Rose Bengal Lactone (CAS 4159-77-7) and Rose Bengal diesters such as Rose Bengal diacetate (CAS 61738-01-0) and Rose Bengal dipropanoate (CAS 61738-01-0).

In some embodiments, the PS is a phenothiazine dye such as a methylene blue dye, for example, methylene blue chloride.

The Polymer:

Herein throughout, the term “polymer” encompasses substances composed of a plurality of repeating structural units (backbone units) covalently connected to one another. The term “polymer” as used herein encompasses organic and inorganic polymers and further encompasses one or more of a homopolymer, a copolymer or a mixture thereof (a blend). The term “homopolymer” as used herein describes a polymer that is made up of one type of repeating backbone units. The term “copolymer” as used herein describes a polymer that is made up of more than one type of backbone units and hence is composed of heterogenic backbone units. The heterogenic backbone units can differ from one another by the pendant groups thereof. The heterogenic backbone units can be arranged in the polymer in any order with respect to one another.

The term “polymer” as used herein throughout is also referred to herein interchangeably as a “polymeric material”.

The polymers used in the context of any one of the embodiments of the present invention, and any combination thereof, are thermoplastic polymers (and/or co-polymers).

The phrase “thermoplastic polymer” describes a polymeric material (a polymer and/or co-polymer) that softens (e.g., becomes pliable or moldable) above a certain temperature and hardens (solidifies) again upon cooling.

In some embodiments of the present invention, the thermoplastic polymer is characterized by a melting transition temperature (Tm) that is lower than the decomposition temperature of the photosensitizer incorporated therein.

In some embodiments, the thermoplastic polymer is characterized by a Tm lower than 400° C.

In some embodiments, the thermoplastic polymer is characterized by a Tm lower than 300° C., lower than 250° C., or lower than 200° C.

Exemplary thermoplastic polymers that are suitable for use in the context of the present embodiments include, but are not limited to, polyolefins, polyesters, polycarbonates, polyurethanes, polyether urethanes, polyether carbonates, polyester carbonates, polyester urethanes, polyanhydrides, polyamides, polyacrylates, and polymethacrylates, any combination of the forgoing and any copolymers thereof.

Combinations of the thermoplastic polymer and the photosensitizer are made by selecting a thermoplastic polymer exhibiting a Tm as described hereinabove. This is due to the unique process described herein, which involves mixing the photosensitizer and the polymer upon melting the polymer (heating a mixture at a temperature that is equal to or higher than the Tm of the polymer).

In some embodiments, the Tm of the polymer is lower by about 50° C. of the decomposition temperature of the PS, and in some embodiments it is lower by 100° C. and even more of the decomposition temperature of the PS. By selecting a polymer with such a Tm, decomposition of the PS during the preparation of the composition is avoided or at least substantially reduced.

In some embodiments, the thermoplastic polymer as described herein is a non-soluble thermoplastic polymer.

Herein throughout, and with reference to any one of the embodiments described herein and any combination thereof, the phrase “non-soluble thermoplastic polymer” describes a thermoplastic polymer which is non-soluble in any organic solvent, including polar and non-polar solvents, in water, and in any combination of an organic solvent and water, at least at room temperature and ambient pressure.

The phrase “organic solvent” describes polar, non-polar, protic, aprotic, low boiling and high boiling organic substances that are commonly used for preparing solutions, preferably solutions of organic polymers. By “solution” it is meant that the organic solvent solvates at least 50%, preferably at least 80%, or at least 90% of the polymer.

Common organic solvents include, but are not limited to, alkanes (e.g., of 1-10 carbon atoms), haloalkanes (e.g., of 1-10 carbon atoms), cycloalkanes, benzene, benzene substituted by one or more of alkyl and halo substituents, heteroatomic compounds such as pyridine or pyrimidine, ethers, carboxylic acids of 1-10 carbon atoms, alcohols of 1-10 carbon atoms, optionally substituted by e.g., halo substituents, ketones of 1-10 carbon atoms, amides (e.g., of 1-4 carbon atoms) and sulfoxides (e.g., of 1-4 carbon atoms).

Representative examples of organic solvents include, but are not limited to, dichloromethane, chloroform, hexane or a mixture of hexanes (aka as petroleum ether), heptanes, benzene, toluene, xylene, cyclohexane, pentane, dioxane, diethyl ether, tetrahydrofuran, ethyl acetate, acetone, dimethylformamide, dimehylsufoxide, acetonitrile, nitromethane, methanol, ethanol, propanol, butanol, acetic acid, isoproanol, formic acid, ethylene chloride, pyridine, methyl isobutyl ketone, dimethylketone, isooctane, octanol, carbon tetrachloride, diethylamine, triethylamine, t-butyl methyl ether, t-butanol, acetone, isobutyl alcohol, isoamyl alcohol, butyl acetate, diethyleneglycol dimethyl ether, benzonitrile, propanoic acid, acetic anhydride, ethylene glycol, diethylene glycol, dichlorobenzene, chlorobenzene, trifluoroethanol, trifluoroacetic acid, and tetrachloroethylene.

By “non-soluble” it is meant, for example, that when 1 gram of the polymer is mixed with 100 ml of the solvent (organic solvent, as described herein, water or a mixture thereof), more that 90% (by weight) of the polymer remains as solid in the mixture, and no more than 10% (by weight) of the polymer is solvated. In some embodiments, more than 95%, or more than 99%, or more than 99.9% of the polymer, by weight, remains as solid, unsolvated, in the mixture. In some embodiments, no more than 5%, or no more than 2%, or no more than 1%, or no more than 0.5, or no more that 0.1%, by weight, is solvated in a solvent as described herein.

The solubility of polymers in various solvents can be determined based on known parameters (e.g., The Hildebrand solubility parameter (δ) or the Hanson solubility parameters) or can be extracted from public databases (e.g., the “Plastic Technology Handbook”).

Exemplary non-soluble thermoplastic polymers which are suitable for use in the context of the present embodiments include, but are not limited to, non-soluble polyolefins such as polyethylene, polypropylene, polybutylene, copolymers of two or more of polyethylene, polypropylene and polybutylene, poly(vinyl chloride), poly(vinylidene chloride), polytetrafluoroethylene, poly(methylmethacrylate), poly(vinyl acetate), cis-polyisoprene and polychloroprene, and any co-polymers of the foregoing.

Embodiments of the present invention encompass ultra-high-molecular-weight polymer or co-polymer, ultra-low-molecular-weight polymer or co-polymer, high-molecular-weight polymer or co-polymer, high-density polymer or co-polymer, high-density cross-linked polymer or co-polymer, cross-linked polymer or co-polymer, medium-density polymer or co-polymer, linear low-density polymer or co-polymer, low-density polymer or co-polymer, and very-low-density polymer or co-polymer of any of the above-described polymers or copolymers thereof. Embodiments of the present invention also encompass halogenated derivatives of any of the above-described polymers or copolymers, as long as these are non-soluble and thermoplastic.

In some embodiments, the polymer is a polyethylene and in some embodiments it is polypropylene.

The polyethylene can be ultra-high-molecular-weight polyethylene (UHMWPE), ultra-low-molecular-weight polyethylene (ULMWPE), high-molecular-weight polyethylene (HMWPE), high-density polyethylene (HDPE), high-density cross-linked polyethylene (HDXLPE), cross-linked polyethylene (PEX or XLPE), medium-density polyethylene (MDPE), linear low-density polyethylene (LLDPE), low-density polyethylene (LDPE), very-low-density polyethylene (VLDPE) or halogenated polyethylene such as chlorinated polyethylene (CPE).

The polyprolylene can be atactic or isotactic, and can also be of any density and/or molecular weight and/or cross-linking, as described herein.

In some embodiments, the polymer is extrudable, that is, it can undergo an extrusion process (e.g., subjected to heat and shear forces, as described herein) without decomposition.

In some embodiments, any of the polymers, polymer mixtures or polymeric matrices made therefrom are photopermeable, or light-transmitting, that is, light (at least at a wavelength absorbed by a PS incorporated therein) can permeate therethrough and thus activate the photosensitizer (PS).

The Process:

According to an aspect of some embodiments of the present invention there is provided a process of preparing a composition which comprises a polymeric matrix which is comprised of a thermoplastic polymer as described herein and a photosensitizer incorporated in the polymeric matrix, the process being effected by heating a solid mixture which comprises the photosensitizer and the thermoplastic polymer to a temperature equal to or higher than a Tm of the polymer, to thereby obtain a fluid mixture of the polymer and the photosensitizer; and cooling the fluid mixture, thereby obtaining the composition.

In some embodiments, the solid mixture comprises the polymer and the PS at a weight ratio that ranges from 1000:1 to 1:1, or from 1000:1 to 10:1 or from 1000:1 to 100:1 or from 500:1 to 50:1, or from 200:1 to 10:1 or from 200:1 to 50:1, or from 150:1 to 50:1, including any intermediate values and subranges therebetween. In exemplary embodiments, the weight ratio is 100:1 (polymer to PS).

As used herein, “a temperature equal to or higher a Tm of the polymer” means a temperature which is about (+/−10%) the Tm of the polymer, or is higher than the Tm of the polymer by e.g., 10, 15, 20, 25, 30 or up to about 50° C., or higher, including any intermediate values and subranges therebetween.

The heating may be performed at once, that is, the mixture is heated to the indicated temperature, and is maintained at that temperature for a certain time period, or it can be performed gradually, by heating the solid mixture first to a temperature that is lower than or equal to the Tm of the polymer, and then elevating the temperature, gradually or at once, to a temperature that is equal to or higher than the Tm of the polymer, respectively, as long as that at some point of the process the temperature is equal to or higher than the Tm of the polymer.

In some embodiments, heating is performed while mixing the mixture, at least during a certain time period during heating.

In some embodiments, the mixing is effected by a method which generates shear forces.

As used herein and in the art, “shear force” refers to a force which causes a stress in a material in a direction which is parallel to a cross-section of the material.

It is to be appreciated that movement of fluids over a solid surface characteristically incurs a shear force.

According to some embodiments, mixing is performed in such a way as to maximize passage of the mixture over solid surfaces. Optionally, solid components with large surface areas (e.g., a screw, a propeller) are utilized to increase shear force.

Optionally, shear forces are generated by a compounder, such as, without limitation, an extruder, an internal mixer (a Banbury® mixer), a co-kneader, and/or a continuous mixer, etc.

In some embodiments, the shear forces and mixing time are selected such that the photosensitizer is essentially evenly dispersed throughout the polymeric matrix.

According to some embodiments, mixing is effected by rotation of a screw. The screw is optionally in a barrel (e.g., the barrel forming a closed container). The barrel may optionally be heated (e.g., by an electric heater) in order to effect heating along with mixing. Alternatively or additionally, the screw may optionally be heated (e.g., by a flow of heated fluid inside the screw) in order to effect heating along with mixing.

In some of any of the embodiments described herein, the mixing is effected by rotation of a screw in an extruder.

An extruder typically comprises a heated barrel containing rotating therein a single or multiple screws. When more than a single screw is used, the screws may be co-rotated or counter-rotated. Screws may be intermeshing, or non-intermeshing. The extrusion apparatus may be a single extruder or combinations of extruders (such as in tandem extrusion) which may be any one of the extruders known in the plastics industry, including, without limitation, a single screw extruder, a tapered twin extruder, a tapered twin single extruder, a twin screw extruder, a multi-screw extruder.

In some embodiments, the extruder is a single screw extruder.

In some embodiments, the process comprises heating the solid mixture while mixing the mixture (e.g., by means of an extruder, as described herein). In some embodiments, the extrusion is performed at a temperature that ranges from 100 to 400° C. In some embodiments, the extrusion is performed at a temperature at which the mixture is maintained as a fluid (e.g., liquid) mixture, which temperature depends mainly of the Tm of the polymer used.

In some of these embodiments, the heating is performed at the feed of an extruder at a first temperature, to obtain the fluid mixture, then the fluid mixture is pushed through the extruder while being heated at a second temperature.

In some embodiments, the first temperature is higher than the second temperature. In some embodiments, the first temperature is lower than a second temperature.

In exemplary embodiments, the extrusion is performed at a temperature that ranges from 100 to 200° C.

In some embodiments the extrusion is continuous. In some embodiments, the extrusion is pulsatile.

The shape and dimensions of the composition can be determined by the shape of the die of the extrusion and/or by selecting a pulsatile or continuous extrusion. Without being bound to any particular theory, it is assumed that by heating and optionally mixing the solid mixture of the polymer and the photosensitizer, as described herein, the photosensitizer dissolves or is dispersed in the melted polymer, whereby the mixing further facilitates the dispersion of the Ps within the polymeric matrix such than an even dispersion is obtained.

In some embodiments, the solid mixture (prior to softening or melting the polymer) and the fluid mixture (subsequent to softening the polymer) consist essentially of the polymer and the photosensitizer.

In some embodiments, both the solid mixture (prior to softening the polymer) and the fluid mixture (subsequent to softening the polymer) are devoid of a surfactant and/or an organic solvent (e.g., an organic solvent as defined herein).

Herein throughout, “devoid of” means less than 1 weight percent, less than 0.01 weight percent, less than 0.005 weight percent, less than 0.0001 weight percents, or null, of the surfactant, the organic solvent, or any other agent discussed herein in this context, with respect to the total weight of the composition.

The term “surfactant” as used herein encompasses amphiphilic compounds having soap-like properties, namely, having a hydrophilic head and a hydrophobic tail. This term encompasses nonionic, zwitterionic, cationic, and anionic surfactants, as these terms are known in the art. Representative examples include potassium salts or ammonium salts of fatty acids, or of hydrophobic sulfonic acids, or of hydrophobic polysulfones. In some embodiments, the surfactant is any of the surfactants as described in WO 99/49823.

In some embodiments, a process as described herein further comprises, prior to or subsequent to the cooling, subjecting the fluid mixture or the composition obtained by the process to further processing steps or procedures, so as to provide the composition with a desired shape, dimensions, surface area, and the like. Such processing procedures include, for example, cutting, molding (e.g., injection molding, compression molding, blow molding), thermal forming, fiber spinning, wool production and/or drawing.

In some embodiments, the additional processing is performed without an addition of an organic solvent and/or surfactants.

Without being bound by any particular theory, it is assumed that once the polymer softens (or melts), upon heating, the PS is dispersed therein, presumably homogeneously, and remains dispersed in the polymer during the entire process (e.g., during extrusion and/or subsequent cooling and/or processing).

This process allows to efficiently incorporate a PS in a polymeric matrix that is made of non-soluble polymers, which are otherwise difficult to process and hence require the use of additional agents such as surfactants, or any other agents.

The process described herein is advantageously performed without the addition of such agents and hence is more cost-effective, and further circumvents the need to remove these agents or otherwise dispose excess of such agents.

The process described herein is advantageously performed without the addition of organic solvents, and is hence devoid of the toxic effects associated with handling organic solvents, of the costly procedures associated with disposal of organic waste, of possible environmental adverse effects associated with using organic solvents, and of many more disadvantages associated with performing small- and large-scale processes using organic solvents.

The process described herein is further advantageous as it results in a composition which is devoid of additional agents such as surfactants and is devoid of residual amounts of organic solvents, as described herein.

The Composition:

According to an aspect of some embodiments of the present invention there is provided a composition comprising a polymeric matrix comprised of a thermoplastic polymer as described herein and a photosensitizer incorporated in the polymeric matrix.

According to an aspect of some embodiments of the present invention there is provided a composition comprising a polymeric matrix comprised of a thermoplastic polymer and a photosensitizer incorporated in the polymeric matrix, the composition being prepared by a process as described herein in any one of its respective embodiments and any combination thereof.

According to some embodiments of the present invention, the composition as described herein is devoid of a surfactant and/or a residual amount of an organic solvent, as defined herein.

Herein throughout, “residual amount” means less than 0.01 weight percent, less than 0.005 weight percent, less than 0.0001 weight percents, or null, of the organic solvent, with respect to the total weight of the composition.

Herein throughout, the phrase “polymeric matrix” describes matrix made of a polymeric material, as described herein in any of the respective embodiments and any combination thereof.

The polymeric matrix can be in any pseudo-two-dimensional or three-dimensional form, including, for example, a fiber, a fibrous network, a rod, a tube, a film, a thin film, a pellet, a bead, a granule, a sheet, and the like, each may be further in various shapes and dimensions.

In some embodiments, when the composition is prepared by a process as described herein, in which a fluid mixture is subjected to extrusion, the shape and dimensions of the matrix can be determined by the die of the extruder and/or by further processing steps subsequent to the extrusion.

In some of any of the embodiments described herein, the polymeric matrix is comprised of the thermoplastic polymer (or copolymer) as described herein.

In some of any of the embodiments described herein, the polymeric matrix comprises the thermoplastic polymer (or copolymer) as described herein.

In some of any of the embodiments described herein, the polymeric matrix comprises the thermoplastic polymer (or copolymer) as described herein, in an amount of at least 80 weight percents, or at least 90 weight percents, or at least 95 weight percents, or at least 99 weight percents of the total weight of the composition.

In some embodiments, the polymeric matrix consists essentially of the non-soluble thermoplastic polymer (or copolymer) as described herein.

In some embodiments, the composition consists essentially of the thermoplastic polymer and the photosensitizer.

In some of any of these embodiments, the thermoplastic polymer is a non-soluble thermoplastic polymer, as described herein in any of the respective embodiments and any combination thereof.

In some of any of the embodiments described herein, the amount of the photosensitizer in the polymeric matrix is in the range of from about 0.1 weight percent to about 10 weight percents of the total weight of the composition.

In some of any of the embodiments described herein, the amount of the photosensitizer in the polymeric matrix is in the range of from about 0.1 weight percent to about 5 weight percents, or from about 0.5 weight percent to about 5 weight percents, or from about 0.1 weight percent to about 2 weight percents, or from about 0.5 weight percent to about 1.5 weight percents, including any subranges and intermediate values therebetween, or is about 1 weight percent, of the total weight of the composition.

In some of any of the embodiments described herein, the composition further comprises one or more additional ingredients (none of which is a surfactant or a residual organic solvent, as described herein), such as, but not limited to, plasticizers, anti-oxidants, preservatives, additional anti-microbial agents, including antibiotics, flavoring agents, odoriferous agents, oxygen carrier molecules, and the like.

In some embodiments, the additional agent is selected so as to not interfere with the photochemical properties of the PS, and is other than a surfactant and an organic solvent, as these terms are defined herein.

In some of any of the embodiments described herein, the composition is devoid of a chemiluminescent agent.

In some of any of the embodiments described herein, the composition is such that the photosensitizer exhibits an activity as described herein, upon exposure to light in an oxygen-containing environment, as described herein. In some of any of the embodiments described herein, the composition is such that the photosensitizer does not exhibit an activity as described herein, when it is not exposed to light and/or when in an oxygen-deficient environment, as described herein.

According to some of any of the embodiments described herein, the composition is such that most or substantially all of the PS is incorporated within the polymeric matrix. That is, the amount of photosensitizer that is present on an external surface of the polymeric matrix is lower than 20%, or lower than 15%, or lower than 10%, or lower than 5%, or even lower, being 1% or less, by weight, of the total weight of the PS.

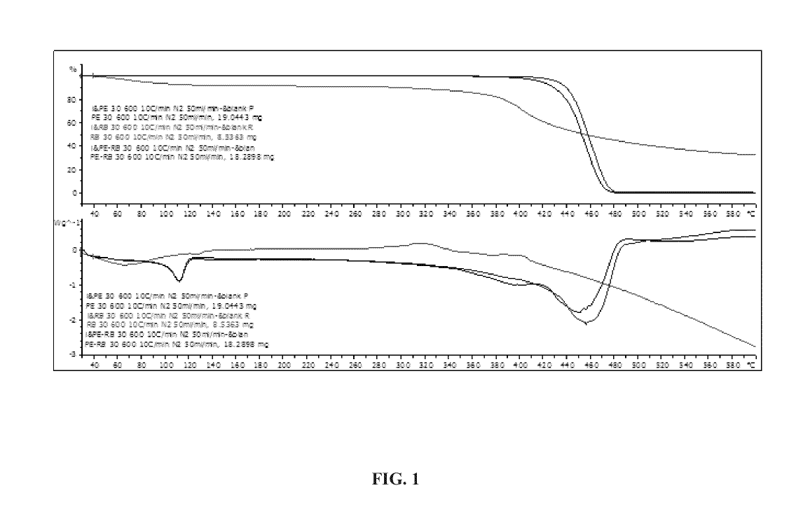

Reference can be made in this regard to FIGS. 1-9C, where SEM measurements show that the surface of exemplary polymeric matrices according to some embodiments of the present invention remained substantially unchanged upon incorporation of the PS, EDS measurements show that the elements on the external surface of the composition are essentially solely of the polymer used for forming the polymeric matrix and TGA analyses demonstrate that thermal stability of the polymeric matrices does not decrease upon incorporation of the PS.

In some embodiments, a composition as described herein is such that upon immersing a sample of the composition in a large excess of water or an aqueous solution for 24 hours, less than 10%, or less than 5%, or even less than 1%, by weight, of the photosensitizer leaches out of the composition.

In some embodiments, the sample is immersed in water or aqueous solution at a ratio of at least 1:10, or 1:100, or 1:1000, by weight.

In some embodiments, the pH of the aqueous solution in which a sample is immersed ranges from 4 to 8.

In some embodiments, the composition is such that a photosensitizing activity of the PS is maintained upon illuminating the composition for at least 10 days.

In some embodiments, at least 80% of the photosensitizing activity is maintained upon exposing the composition to illumination by a luminescent lamp for 10 days. In some embodiments, at least 90%, at least 95%, at least 99% and even 100% of the photosensitizing activity is maintained under these conditions.

In some of any of the embodiments described herein, the amount of PS that leaches from the composition is measured by spectroscopic means (e.g., UV/Visible spectrometer).

In some of any of the embodiments described herein, the photosensitizing activity of the PS is measured by measuring an antimicrobial activity of the composition during a certain time period, as exemplified in Example 4 of the Examples section that follows. Herein throughout, the terms “composition” and “composition-of-matter” are used interchangeably.

Antimicrobial Activity:

In some embodiments, a composition as described herein, in any of the respective embodiments as any combination thereof, exhibits an antimicrobial activity as defined herein, and is also referred to as an antimicrobial composition.

Herein throughout, the phrase “antimicrobial activity” or “antimicrobial effect” refers to effecting death, eradication, elimination, reduction in number, reduction of growth rate, and/or change in population distribution of one or more species of a microorganism.

In some embodiments, an antimicrobial activity is represented by reducing the number of microorganisms (e.g., colony forming units), for example, reducing the number of microorganisms by at least 20%, or by at least 50%, at least 70%, at least 80%, at least 90%, at least 95% and even by 99% or 100% (complete or nearly complete eradication of the microorganisms).

The phrase “microorganism” as used herein describes any microbial species, including unicellular and multicellular living forms, and can be, for example, a bacterium, a virus, a fungus, a protozon, an alga, and the like. In some embodiments, the microorganism is a pathogenic microorganism, as defined herein.

Herein throughout, the phrase “pathogenic microorganism” is used to describe any microorganism or microbial species, as described herein, which can cause a disease or infection in a higher organism, such as any animals grown for commercial or recreational purposes, fish, poultry, insects (e.g., bees) and mammals. In some embodiments, the pathogenic microorganism is such that causes diseases and/or adverse effects in humans.

A pathogenic microorganism, or microbial species, may belong to any family of organisms such as, but not limited to, bacteria, virus, yeast, fungi, algae, protozoa, and other parasites.

Non-limiting examples of pathogenic microorganism include Plasmodium falciparum and related malaria-causing protozoan parasites, Acanthamoeba and other free-living amoebae, Aeromonas hydrophila, Anisakis and related worms, Ascaris lumbricoides, Bacillus cereus, Campylobacter jejuni, Clostridium botulinum, Clostridium perfringens, Cryptosporidium parvum, Cyclospora cayetanensis, Diphyllobothrium, Entamoeba histolytica, Eustrongylides, Giardia lamblia, Listeria monocytogenes, Nanophyetus, Plesiomonas shigelloides, Salmonella, Shigella, Staphylococcus aureus, Streptococcus, Trichuris trichiura, Vibrio cholerae, Vibrio parahaemolyticus, Vibrio vulnificus and other vibrios, Yersinia enterocolitica and Yersinia pseudotuberculosis.

Exemplary viruses include, but are not limited to, viruses with single or double-stranded nucleic acid genomes, DNA or RNA viruses and include enveloped as well as some non-enveloped viruses. Viruses that comprise negative single-stranded RNA genomes include, for example, Orthomyxoviridae, Rhabdoviridae, Paramyxoviridae, Bunyaviridae, and Filoviridae. Orthomyxoviridae include the influenza viruses A, B, and C. Rhabdoviridae include rabies virus and vesicular stomatitis virus. Paramyxoviridae include parainfluenza virus of mammals (including mumps virus) and cattle. Bunyaviridae include hantavirus, which causes Korean hemorrhagic fever and hantavirus pulmonary syndrome. Filoviridae include Marburg virus and Ebola virus. Viruses that comprise positive single-stranded RNA genomes include Picornaviridae (non-enveloped), Retroviridae, and Togaviridae, Picornaviridae include polioviruses, coxsackieviruses, hepatitis A virus, and rhinovirus. Retroviridae include, for example, human immunodeficiency virus (HIV), simian immunodeficiency virus (SIV), and equine infectious anemia virus (EIAV). Togaviridae include Semliki Forest virus, yellow fever virus, Dengue virus, tick-borne virus, and rubella virus. Parvovirus (nonenveloped) is the only virus having a single-stranded negative-sense DNA genome. Double-stranded viruses include Papovaviridae, Adenoviridae, Herpesviridae, Poxviridae, and Hepadnaviridae. Papovaviridae include papillomaviruses causing warts and tumors. Adenoviridae include Mastadenovirus and a variety of viruses capable of infecting the respiratory tract. Herpesviridae include herpes simplex 1 and 2, varicella zoster virus, cytomegalovirus, Epstein-Barr virus, human herpesvirus 6, and human herpesvirus 7. Poxviridae include variola and other pox-producing viruses. Hepadnaviridae include human hepatitis B virus.

Exemplary bacteria include, but are not limited to, Gram negative, Gram positive and mycobacteria strains such as Strep. pyogenes (Group A), Strep. pneumoniae, Strep. GpB, Strep. viridans, Strep. GpD (Enterococcus), Strep. GpC and GpG, Staph. aureus, Staph. epidermidis, Bacillus subtilis, Bacillus anthracia, Listeria monocytogenes, Anaerobic cocci, Clostridium spp., Actinomyces spp, Escherichia coli, Enterobacter aerogenes, Kiebsiella pneumoniae, Proteus mirabilis, Proteus vulgaris, Morganella morganii, Providencia stuartii, Serratia marcescens, Citrobacter freundii, Salmonella typhi, Salmonella paratyphi, Salmonella typhi murium, Salmonella virchow, Shigella spp., Yersinia enterocolitica, Acinetobacter calcoaceticus, Flavobacterium spp., Haemophilus influenzae, Pseudomonas aeruginosa, Campylobacter jejuni, Vibrio parahaemolyticus, Brucella spp., Neisseria meningitidis, Neisseria gonorrhoea, Bacteroides fragilis, Fusobacterium spp., Mycobacterium tuberculosis, Mycobaterium smegmatis. Pseudomonas aeruiginosa, and Escherichia coli. Other bacteria include, but are not limited to, other species of Staphylococcus, Enlerlococcus, Slreplococculs, Corynebaclerium, Listeria, Neisseria, and Enterobacteriaceae.

Exemplary fungi include, but are not limited to, fungi of the genus Absidia: Absidia corymbifera; genus Ajellomyces: Ajellomyces capsulatus, Ajellomyces dermatitidis; genus Arthroderma: Arthroderma benhamiae, Arthroderma fulvum, Arthroderma gypseum, Arthroderma incurvatum, Arthroderma otae, Arthroderma vanbreuseghemii; genus Aspergillus: Aspergillus flavus, Aspergillus fumigatus, Aspergillus niger; genus Blastomyces: Blastomyces dermatitidis; genus Candida: Candida albicans, Candida glabrata, Candida guilliermondii, Candida krusei, Candida parapsilosis, Candida tropicalis, Candida pelliculosa; genus Cladophialophora: Cladophialophora carrionii; genus Coccidioides: Coccidioides immitis; genus Cryptococcus: Cryptococcus neoformans; genus Cunninghamella: Cunninghamella sp.; genus Epidermophyton: Epidermophyton floccosum; genus Exophiala: Exophiala dermatitidis; genus Filobasidiella: Filobasidiella neoformans; genus Fonsecaea: Fonsecaea pedrosoi; genus Fusarium: Fusarium solani; genus Geotrichum: Geotrichum candidum; genus Histoplasma: Histoplasma capsulatum; genus Hortaea: Hortaea werneckii; genus Issatschenkia: Issatschenkia orientalis; genus Madurella: Madurella grisae; genus Malassezia: Malassezia furfur, Malassezia globosa, Malassezia obtusa, Malassezia pachydermatis, Malassezia restricta, Malassezia slooffiae, Malassezia sympodialis; genus Microsporum: Microsporum canis, Microsporum fulvum, Microsporum gypseum; genus Mucor: Mucor circinelloides; genus Nectria: Nectria haematococca; genus Paecilomyces: Paecilomyces variotii; genus Paracoccidioides: Paracoccidioides brasiliensis; genus Penicillium: Penicillium marneffei; genus Pichia, Pichia anomala, Pichia guilliermondii; genus Pneumocystis: Pneumocystis carinii; genus Pseudallescheria: Pseudallescheria boydii; genus Rhizopus: Rhizopus oryzae; genus Rhodotorula: Rhodotorula rubra; genus Scedosporium: Scedosporium apiospermum; genus Schizophyllum: Schizophyllum commune; genus Sporothrix: Sporothrix schenckii; genus Trichophyton: Trichophyton mentagrophytes, Trichophyton rubrum, Trichophyton verrucosum, Trichophyton violaceum; and of the genus Trichosporon: Trichosporon asahii, Trichosporon cutaneum, Trichosporon inkin, Trichosporon mucoides.

In some embodiments, the composition as described herein exhibits an antibacterial activity, as defined herein, when contacted with a medium (e.g., an aqueous medium) that comprises bacteria at a concentration of more than 10⁴ or more than 10⁵ or more than 10⁶ CFU/mL, and even at higher concentration.

In some embodiments, a composition as described herein exhibits an antibacterial activity, as defined herein, when contacted with a medium (e.g., an aqueous medium) that comprises bacteria, at concentration of the composition in the medium which is 0.01 gram/mL or higher.

The compositions as described herein exhibit an antimicrobial activity as described herein upon being subjected to conditions under which the PS generates reactive oxygen species.

By “conditions under which the PS generates reactive oxygen species” it is meant: (i) an oxygen-containing environment, which contains oxygen in an amount that when contacted with the PS, the PS converts the oxygen into reactive oxygen species in an amount that exhibits antimicrobial activity, as described herein; and (ii) exposure to light at a wavelength that excites the PS and turns it to be active in generating reactive oxygen species in the presence of oxygen.

In some embodiments, the compositions as described herein exhibit an antibacterial activity upon being exposed to light in an oxygen-containing environment.

“An oxygen-containing environment” includes air, fluids such as water or aqueous solution or reservoirs, bodily fluids (e.g., blood, sweat), and any other oxygen-containing fluids.

“Exposure to light” can be made spontaneously, by having the composition being exposed to, for example, daylight, sunlight, or an environmental light source such as a lamp, or can be made by illumination (irradiation), by exposing the composition to a light emitting device.

In one embodiment, the composition may be exposed to a light source which emits radiation having a wavelength, or a range of wavelengths, within the range of wavelengths absorbed by the photosensitizer, preferably near or corresponding to the wavelength of maximum absorption of the photosensitizer (,max).

In general, any light source that emits light of an appropriate wavelength may be used. The source of light may be any device able to generate or emit monochromatic or polychromatic light, coherent or incoherent light, especially visible white light. Examples include, without limitation, a fluorescent light source, a luminescent light source, a laser, a light emitting diode, an arc lamp, a halogen lamp, and an incandescent lamp.

The light may be produced by any suitable art-disclosed light emitting devices for use in photodynamic disinfection such as lasers (e.g., non-thermal lasers or the like), light emitting diodes (“LEDs”), incandescent sources, fluorescent sources, or the like.

Depending on the amount of photosensitizer and the power of the light emitting device(s), the exposure to light may only require a short period of time such as from about 15 seconds to less than about 5 minutes, or from about 15 seconds to about two minutes, or from about 15 seconds to about 90 seconds, or from about 30 seconds to 60 seconds.

The light energy provided during each cycle of exposure to light may range from about 1 J/cm² to about 100 J/cm², from about 1 J/cm² to about 50 J/cm², from about 1 J/cm² to about 25 J/cm², from about 5 J/cm² to about 20 J/cm², and from at about 6 J J/cm² to about 12 J/cm². Higher and lower values are also contemplated.

A suitable laser may have a power of from about 1 to about 100 W. Other suitable lasers may have a power of about 1 to about 1000 mW and a beam diameter of from about 1 to about 10 mm. The light dose for laser irradiation is suitably from about 5 to about 333 J cm⁻², preferably from about 5 to about 30 J cm⁻² for laser light.

For white light irradiation, a suitable dose is from about 0.01 to about 100 J/cm², preferably from about 0.1 to about 20 J/cm², more preferably from about 3 to about 10 J/cm². Higher and lower values are also contemplated.

Without limitations, the following are examples of light sources and their respective exemplary wavelengths and/or power outputs that may be suitable for use in the present invention:

Helium neon (HeNe) gas laser (e.g. 633 nm);

Argon-pumped dye laser (e.g. 500-700 nm, 5 W output);

Copper vapour-pumped dye laser (e.g. 600-800 nm);

Excimer-pumped dye laser (e.g. 400-700 nm);

Gold vapour laser (e.g. 628 nm, 10 W output);

Tunable solid state laser (e.g. 532-1060 nm), including Sd:YAG;

Light emitting diode (LED) (e.g. 400-800 nm);

Diode laser (e.g. 630-850 nm, 25 W output), e.g. gallium selenium arsenide;

Tungsten filament lamp;

Halogen cold light source;

Fluorescent lamp (e.g. 10 to 30 W);

Luminescent lamp;

Chemiluminescent light;

Sonoluminescent light;

Sonodynamic excitation; and

Sunlight.

In some embodiments, the light source is sunlight and exposure to a light source comprises exposure a composition as described herein to sunlight. The exposure can simply comprise placing the composition in an environment that is exposed to sunlight.

The duration of exposure to light should be long enough to ensure an antimicrobial effect, as defined herein.

In some embodiments, the duration of exposure to light is from about one second to about 10 hours, or from about 1 minute to about 5 hours, or from about 1 minute to about 120 minutes.

In some embodiments, for example when the light source is of low intensity such as exposure to natural daylight, the composition is exposed to light for a longer period of time, such as for several days or several hours, for example from about 1 to about 24 hours.

The light may be delivered to the mixture by ambient exposure, or, if necessary or convenient, by use of a directed means such as a fibre optic light source or other known optical devices.

The exposure to light can be repeated several times (e.g., about 2 to about 10, about 3 to about 5, etc.) until a desired effect (e.g., an antimicrobial activity as defined herein) has been reached.

Articles:

The compositions as described herein can form a part of an article-of-manufacturing (also referred to herein simply as “article”).

According to an aspect of some embodiments of the present invention there is provided an article-of-manufacturing comprising any one of the compositions as described herein in any one of the respective embodiments and any combination thereof.

In some embodiments, the composition as described herein forms the article-of-manufacturing per se, or forms at least the major body of the article. In some of these embodiments, the article-of-manufacturing consists of the composition, and the composition is shaped and/or processed as desired to form the article-of-manufacturing.

In some embodiments, the composition forms a part of the article-of-manufacturing, and joined together with other components to form the article.

The articles-of-manufacturing or the composition described herein therein can be provided in a variety of forms and shapes. For example, the composition can be formed as a coating, a film, a fiber, or a pellet, and be incorporated as such to the article.

In some embodiments, the composition is in a form of fibers, which can form woven, knitted or non-woven articles such as textile products or medical devices such as patches, meshes, bandage, gauzes, wipes, wound dressings, surgical drapes, and the like.

In some embodiments, the article is formed of the composition per se, formed and shaped as a self-supporting film, vessel or container, for example, for coating, packaging or storage of medical devices, drugs, cosmetic products, agricultural products, computer components, food products, beverages and the like.

In some embodiments, the articles or the compositions are shaped as mats, or tubes, or valves. In some embodiments, the articles or compositions are shaped as laminates, for coating or covering or wrapping substrates such as wood, glass, metals, or plastic substrates.

In some embodiments, the articles or the compositions are for underwater use and/or for water treatment, and can take the form of, for example, rods, pellets or beads, which may be used as is or form a bed of a column of such pellets or beads.

In some embodiments, the article-of-manufacturing is such that benefits from the antimicrobial activity exhibited by the composition. For example, the article-of-manufacturing is an article which is susceptible to, or promotes, microbial harboring and/or growth.

Exemplary articles-of-manufacturing comprising, made of, incorporating and/or coated by a composition as described herein include, but are not limited to, medical devices, water conduits, water or other fluid storage vessels, pipes, tubes, valves, filters, feeders, fittings, seals, basins, packages (e.g., food packages or wraps, medical devices packages such as contact lens cases, computer components packages and the like), packaging materials, food processing facilities, dining facilities (e.g., cloths, dining tables), toys (e.g., pool toys), sanitation products (e.g., toilette seats and water tanks, wash basin), door handles, door knobs, light switches and covers, sink handles, dental instruments, computer keyboards, computer input devices, food containers, beverage containers (e.g., beverage bottles), diaper lining, sanitary pad lining and casing, ropes, carpets, piping systems, car batteries, insulated electrical cables, filters for fluids, detergent containers, constructions products (e.g., gutter and roofing sheets, shingles, wooden shakes, tiles), tubing, fabrics and textile products, household containers and reservoirs, air conditioning ducts, and more.

As used herein, the phrase “medical device” includes any material or device that is used on, in, or through a subject's body, for example, in the course of medical treatment (e.g., for a disease or injury). Medical devices include, but are not limited to, such items as medical implants, wound care devices, drug delivery devices, contact lenses and body cavity and personal protection devices.

Exemplary medical implants include, but are not limited to, urinary catheters, intravascular catheters, dialysis shunts, wound drain tubes, skin sutures, vascular grafts, implantable meshes, intraocular devices, heart valves, and the like. Wound care devices include, but are not limited to, general wound dressings, biologic graft materials, tape closures and dressings, and surgical incise drapes. Drug delivery devices include, but are not limited to, needles, skin patches, mucosal patches, medical sponges and cosmetic applicators. Body cavity and personal protection devices, include, but are not limited to, tampons, sponges, surgical and examination gloves, toothbrushes and other dental instruments. Birth control devices include, but are not limited to, intrauterine devices (IUDs), diaphragms and condoms.

Other exemplary medical devices include, for example, stethoscopes and similar articles used when evaluating a patient's health condition.

In embodiments where the composition is formed and/or shaped to provide an article-of-manufacturing, the articles can be any article that is made of the non-soluble thermoplastic polymer as described herein.

For example, articles made of polyethylele include, but are not limited to, moving machine parts, bearings, gears, artificial joints, bulletproof vests, milk jugs, liquid laundry detergent bottles, outdoor furniture, margarine tubs, portable gasoline cans, water drainage pipes, grocery bags, packaging film, sacks, gas pipes and fittings, squeeze bottles, milk jug caps, retail store bags, stretch wrap in transporting and handling boxes of durable goods, a household food covering.

Exemplary articles made of polypropylene include, but are not limited to, reusable plastic food containers, microwave- and dishwasher-safe plastic containers, diaper lining, sanitary pad lining and casing, ropes, carpets, plastic moldings, piping systems, car batteries, insulation for electrical cables filters for gases and liquids, heat-resistant medical equipment, and stationery folders and packaging.

Articles made of polyvinyl chloride (PVC) include construction elements such as for vinyl siding, drainpipes, gutters and roofing sheets, hoses, tubing, electrical insulation, coats, jackets and upholstery, water beds and pool toys.

Articles made of teflon include a coating for non-stick cookware, containers and pipes that come in contact with reactive compounds, gears, bearings, and bushings.

Articles made of polycarbonates include, for example, beverage bottles.

For any of the articles-of-manufacturing described herein, it is preferred that the article is designed such that the composition included therein, or forming it, can be exposed to light. In some embodiments, the articles are designed such that at least the portion thereof which contains the composition is exposed to light or is photopermeable (as defined herein) to radiation at a wavelength that activates the PS.

Applications:

In some embodiments, any of the article-of-manufacturing or the composition as described herein is a self-disinfecting or self-sterilizing article, when subjected to conditions in which the photosensitizer generates reactive oxygen species in the presence of oxygen, as described herein.

According to an aspect of some embodiments of the present invention there is provided a method of disinfecting or sterilizing any of the article-of-manufacturing or the composition as described herein, which is effected by exposing the article-of-manufacturing or the composition to conditions in which the photosensitizer generates reactive oxygen species, as described herein.

According to an aspect of some embodiments of the present invention there is provided a method of disinfecting or sterilizing the article-of-manufacturing or the composition as described herein, which is effected by exposing the article-of-manufacturing or the composition to light, as described herein, in the presence of oxygen (e.g., an oxygen-containing environment).

For example, an article-of-manufacture as described herein can be a medical device, such as an implantable medical device, or a medical device used in surgery, and the medical device is disinfected prior to its use by the method as described herein.

In another example, the article-or-manufacture is a package or a storage vessel or container of, for example, a medical device, a drug, a cosmetic product, a food product, a beverage, an agricultural product or a construction product, or a computer system component, and the package or vessel or container is disinfected by the method as described herein prior to packaging therein the product. In some of these embodiments, the package or storage vessel or container is photopermeable as defined herein. The package, vessel or container can be disinfected periodically, as long as it is in use, so as to provide continued disinfection of the environment of the product included therein.

The articles-of-manufacturing or compositions as described herein can further be utilized in a method of disinfecting or sterilizing a substrate, which is effected by contacting the substrate with the article-of-manufacturing or the composition, and exposing the article-of-manufacturing or the composition to conditions in which the photosensitizer generates reactive oxygen species, as described herein, or exposing the article-of-manufacturing or the composition to light, as described herein, in the presence of oxygen (e.g., an oxygen-containing environment).

By “contacting” it is meant bringing the substrate and the composition or article to an intimate contact or in close proximity (e.g., of up a few centimeters).

In some embodiments, the contacting is effected for a time period which depends on the amount of microbial load in or on the substrate, the concentration of the PS and the light intensity. In some embodiments, the contacting is effected from about 1 second to about 10 hours, or from about 10 seconds to about 2-3 hours, or from about 1 minute to about 120 minutes or from about 30 minutes to about 120 minutes.

The term “substrate” as used herein, describes any surface, structure, product or material which can support, harbor or promote the growth of a microorganism.

The substrate can be an animate or inanimate substrate.

Animate substrates include, for example, bodily surfaces or cavities, such as, but not limited to, skin, mucosal membranes or cavities, oral cavities, bodily fluids such as blood or portions thereof, and the like.

Body cavities or surfaces include, for example, mouth or oral cavity, nose, ear, rectum, vagina, lung, the entire digestive tract (e.g., throat, esophagus, stomach, intestines, rectum, etc.), gall bladder, bladder, any open wound or the like, as well, as skin, nails, hair, or the like.

When the substrate in an animate substrate, the method as described herein can be used for treating a microbial infection or any other disease or disorder associated with a microorganism in a subject in need thereof.

According to some embodiments, the method as described herein can be utilized for disinfecting an infected bodily area (e.g., an acute or chronic wound, as detailed hereinafter) or for preventing infections in bodily areas that are at risk for being infected, in a subject. The latter include, for example, surgical wounds, acute wounds, ulcers and the like, which are highly susceptible to infections by various microorganisms.

In some embodiments, the method as described herein can be utilized in the treatment of wounds where the reduction of the microbial load in the wound is therapeutically beneficial.

As used herein, the term “subject” includes mammals, preferably human beings at any age which suffer from the pathology (an infection as described herein) or which have been diagnosed as being afflicted by the pathology or which are at risk of being afflicted by the pathology.

The term “wound” is used herein to be construed according to its broadest meaning so as to describe damaged or disturbed skin or mucosal area, whether or not containing devitalized or eschar tissue, and encompasses any type of wound, including, but not limited to, acute wounds, chronic wounds, surgery wounds, burns and the like.

The term “skin lesion” as used herein describes damaged skin and is used interchangeably with the term “wound” throughout the application.

The term “wound area” describes the area adjacent to the wound. This area typically extends from immediately adjacent the wound up to about 30 cm. It is being understood that the inner boundary of the area peripheral to the wound may conform to or parallel the shape of the wound.

The term “acute wound” describes a wound caused by a traumatic abrasion, laceration or through superficial damage, and which eventually heals spontaneously without complications through normal phases of wound healing (such as hemostasis, inflammation, proliferation and remodeling). Acute wounds, however, can often be complicated if become in contact with pathogenic microorganisms that may lead to local infection.

The phrases “surgery wound” and “surgical wound”, which are used herein interchangeably, describe a wound that is formed as a result of a surgical procedure.

Surgical wound infections are common, being 12% of all hospital-acquired infections. The rate of infection varies depending on the type of surgery undertaken. Especially high rates are associated with contaminated surgery, such as colorectal surgery or delayed surgery to traumatic wounds. Surgical wound infections are usually caused by the patient's normal flora or by bacteria from the environment or the skin of hospital staff. The most common microorganism that leads to surgical would infection is Staphylococcus aureus. Other common causative microorganisms include other Gram-negative aerobes, Streptococcus spp. and anaerobes.

The term “burns” describes wounds caused by heat, cold, electricity, chemicals, light, radiation, or friction. Burns can be highly variable in terms of the tissue affected, the severity, and resultant complications. Muscle, bone, blood vessel, and epidermal tissue can all be damaged with subsequent pain due to profound injury to nerve endings. Pathogenic microorganisms that commonly infect burn wounds include, for example, gram-positive bacteria such as methicillin-resistant Staphylococcus aureus (MRSA) and gram-negative bacteria such as Acinetobacter baumannii-calcoaceticus complex, Pseudomonas aeruginosa, and Klebsiella species.

The phrase “chronic wound” describes a wound in which there is no clot formation, normally occurring in patients who are compromised in some fashion and are less likely to heal. Examples of chronic wounds are chronic cutaneous ulcers such as diabetic ulcers, decubitus ulcers (pressure ulcers), and venous ulcers.

The phrase “diabetic ulcers” describes a wound caused by combination of factors associated with diabetes, such as decreased circulation, loss of sensation, structural foot deformities and loss of skin integrity. A diabetic ulcer can be a simple break in the skin, which does not heal in a timely and orderly fashion, or a wound that extends to deep structures and bone. Diabetic ulcers are often the entry points for bacteria and fungal organisms to invade the body, and the cause of limb and life threatening infection, often referred to in the art as diabetic infection. Diabetic infections are usually polymicrobial involving infections caused by multiple aerobic and anaerobic microorganisms. Staphylococcus aureus, beta-hemolytic streptococcus, Enterobacteriaceae, Bacteroides fragilis, Peptococcus, and Peptostreptococcus are exemplary strains that were cultured from diabetic ulcers.

The phrase “decubitus ulcers”, also known as “pressure ulcers”, describe skin lesions caused by variable factors such as: unrelieved pressure; friction; humidity; shearing forces; temperature; age; continence and medication; to any part of the body, especially portions over bony or cartilaginous areas such as sacrum, elbows, knees, ankles etc. Although easily prevented and completely treatable if found early, decubitus ulcers are often fatal and are one of the leading iatrogenic causes of death reported in developed countries. Decubitus ulcers may be caused by inadequate blood supply and resulting reperfusion injury when blood re-enters tissue. The most common organisms isolated from pressure ulcers are Proteus mirabilis, group D streptococci, Escherichia coli, Staphylococcus species, Pseudomonas species, and Corynebacterium organisms.

The phrase “venous ulcer” describes wounds that are thought to occur due to improper functioning of valves in the veins, usually of the legs, causing the pressure in the veins to increase. They are a major cause of chronic wounds, occurring in approximately 30-40% of chronic wound cases. Most venous ulcers are heavily contaminated with bacteria such as Staphylococcus, Eschrichia coli, Proteus and Pseudomonas.

In some embodiments, the method is effected by utilizing an article-of-manufacturing which is a wound dressing. The wound dressing can be designed to have adhesive means for attaching to the wound area. The wound dressing can also have a removable tape or film covering the portion thereof which contains the composition, so as to avoid bleaching of the PS upon exposure to daylight or other light. The tape or film is removed when it is desired to expose the wound dressing to light, as described herein.

In another example, a bodily fluid such as blood, is disinfected outside the body, by passing the fluid through a membrane, filter of column packed with beads comprising the composition as described herein, or adding the composition to a container (photopermeable) comprising the fluid, and exposing the composition to light, as described herein.

In some embodiments, the articles-of-manufacturing or compositions described herein are used for treating a microbial infection in a bodily tissue or cavity as described herein, using the method as described herein.

In some embodiments, the compositions as described herein are used as a pharmaceutical composition which further comprises a pharmaceutically acceptable carrier.

Inanimate substrates include, but are not limited to, water, water reservoirs, aquariums, water treatment systems, which include pipes and tubes, valves, filters, fittings, seals, reservoirs and basins, all of which are susceptible to microbial growth.

Additional non-limiting examples of inanimate substrates include the inner walls of a storage container that is routinely treated with anti-microbial agents, preferably anti-fungal agents, a soil and/or soil enrichment supplements, any agricultural product or crop such as wood, fiber, fruit, vegetable, flower, extract, horticultural crop and any other processed or unprocessed agricultural product or crop which are produced from organic origins such as living plants or animals, a cosmetic product, a building, warehouse, compartment, container or transport vehicle, a dye or a paint and any other materials and industrial compounds which require protection of their surfaces against microbes, moulds and fungi attacks, such as, for example, construction materials.

Such products include, for example, food products, agricultural products, cosmetic products and many more.

In exemplary, non-limiting, embodiments, the method is utilized for disinfecting fluids. The fluid may be placed in or passed through a photopermeable container, made of the composition as described herein or otherwise containing the composition, and the container is exposed to light. Suitable containers include bags, boxes, troughs, tubes or tubing. The light source may be continuous or pulsed. The composition or article may be added directly to the fluid to be treated, or may be flowed into the photopermeable container separately from the fluid being treated, or may be added to the fluid prior to placing the fluid in the photopermeable treatment container. Instead of a container, the fluid may be passed through a filter or membrane in which the composition is embedded, and the composition is exposed to light.

Generally, the compositions and articles described herein can be used in maintaining clean water sources, piping, filters, membranes, containers and reservoirs in households, desalinations plants, drinking fountains, closed-loop cooling towers, air conditioning ducts and related applications. These compositions or articles can further be incorporated into surfaces of objects such as medical devices, as well as pipes and membranes involved in water treatment, water purification and for use in drinking water.

In exemplary embodiments, the substrate comprises water, and the method as described herein is for disinfecting the water. In some embodiments, the method can be effected by placing a composition as described herein, in a form of rods or pellets in a water reservoir to be disinfected. In some embodiments, an article that is in constant contact with the water reservoir is made of, or comprises, the composition as described herein. Such articles include, for example, feeder in aquariums, filters or floats used in circulating water reservoirs such as swimming pools or fountains, and the like.

The antimicrobial properties of the compositions and articles described herein may find application in hospitals and other places where microbiological cleanliness is necessary, for example food processing facilities, dining areas, abattoirs or play areas.

It is to be noted that the articles, substrates and applications described herein are not to be regarded as limiting and that any other articles and substrates which can benefit of the antimicrobial activity of the compositions described herein is contemplated in any one of the relevant embodiments described herein.

It is expected that during the life of a patent maturing from this application additional relevant polymers and/or photosensitizers and/or articles will be developed and the scope of these terms is intended to include all such new technologies a priori.

As used herein the term “about” refers to ±10%.

The terms “comprises”, “comprising”, “includes”, “including”, “having” and their conjugates mean “including but not limited to”.

The term “consisting of” means “including and limited to”.

The term “consisting essentially or means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non limiting fashion.

### Example 1

### Samples Preparation and Characterization

Polymers:

Low-density polyethylene (abbreviated as PE) beads (5 mm diameter) were purchased from Carmel Olefins Ltd, Israel.

Polypropylene (abbreviated as PP) beads (3 mm diameter) were purchased from Carmel Olefins Ltd, Israel.

Photosensitizers (PSs):

Rose Bengal disodium salt (CAS 632-69-9, powder, 95% dye) (abbreviated as RBS) was purchased from Sigma-Aldrich, USA.

Rose Bengal lactone (CAS 4159-77-7, powder, 95% dye) (abbreviated as RBL) was purchased from Sigma-Aldrich, USA.

Methylene blue chloride. 3H₂O (CAS 7220-79-3, powder, 82% dye) (abbreviated as MB) was purchased from Sigma-Aldrich, USA.

Table 1 below presents some data of the PSs tested.

TABLE 1

| | | λmax |
|---|---|---|
| PS | Structural Formula | (nm) |
| MB | 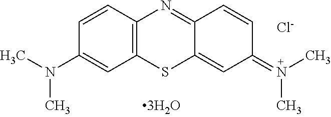 | 665 |
| RBS | 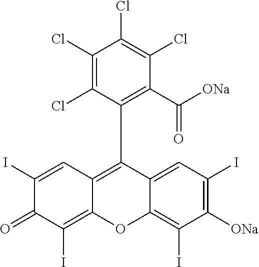 | 544 |
| RBL | 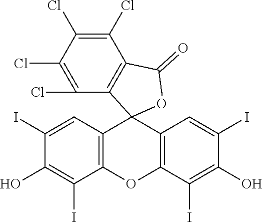 | 557 |

Methods:

Extrusion was performed using an extruder (Allspeeds Ltd, England) under inlet temperature of 80-90° C. and the outlet temperature of 150-200° C.

In an exemplary procedure, a mixture of polymer beads and PS powder were placed in a feed and the extruder was activated to melt the mixture at 43 rpm, then the resulting fluid composition was pushed through a die having a 5 mm round hole or a flat 1×19.6 mm hole. As a result, polymeric rods with having the PS incorporated therein were obtained. The rods were chopped into 3 mm pellets or used as is.

Analytical Procedures:

TGA measurements were performed using the TGA/DSC STAR^{e} System (Mettler Toledo International Inc., operated at 40-600° C. under heating rate of 10° C. per minute in the nitrogen flow of 50 ml per minute.

SEM measurements were performed using a SEM microscope JSM 6510, JEOL, Japan.

EDS analyses were carried out by Noran Microanalysis System supplied with SDD detector connected to the SEM microscope.

### Experimental Results

### A Polymer Composition Comprising Polyethylene and Rose Bengal

1000 grams of polyethylene (PE) beads and 10 grams of Rose Bengal (RBS or RBL) powder were co-extruded in the extruder, as described hereinabove, to produce 5 mm diameter rods. The rods were chopped into 3 mm long pellets having a characteristic Rose Bengal color.

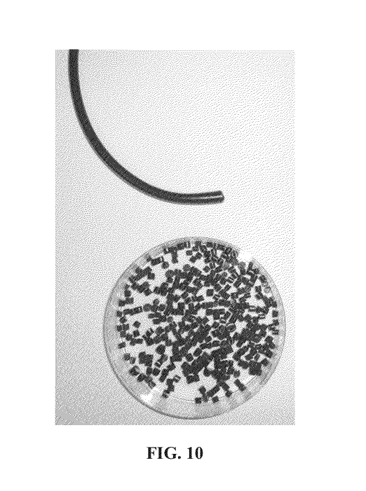

FIG. 10 presents images of the obtained PE-RBS rod (upper image) and the chopped pellets thereof (lower image).

FIG. 1 presents the TGA data of PE (black), RBS (green) and PE-RBS (PE having RBS incorporated therein) (blue). (Upper diagram—mass changes and lower diagram—specific power changes).

It can be seen from the lower graph that RBS alone decompose at 400-410° C. and PE before extrusion decomposes at 450° C. PE-RBS is found to be more stable by 8° to thermal decomposition than PE.

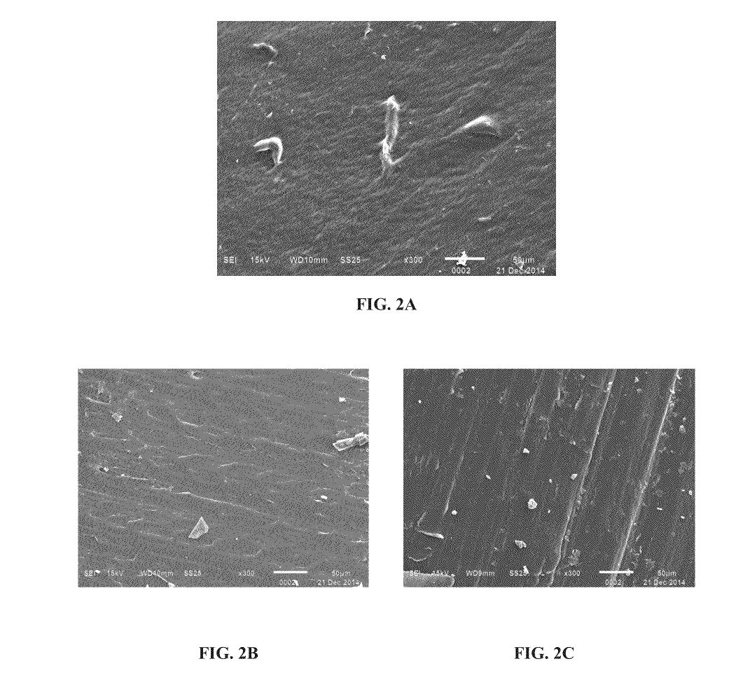

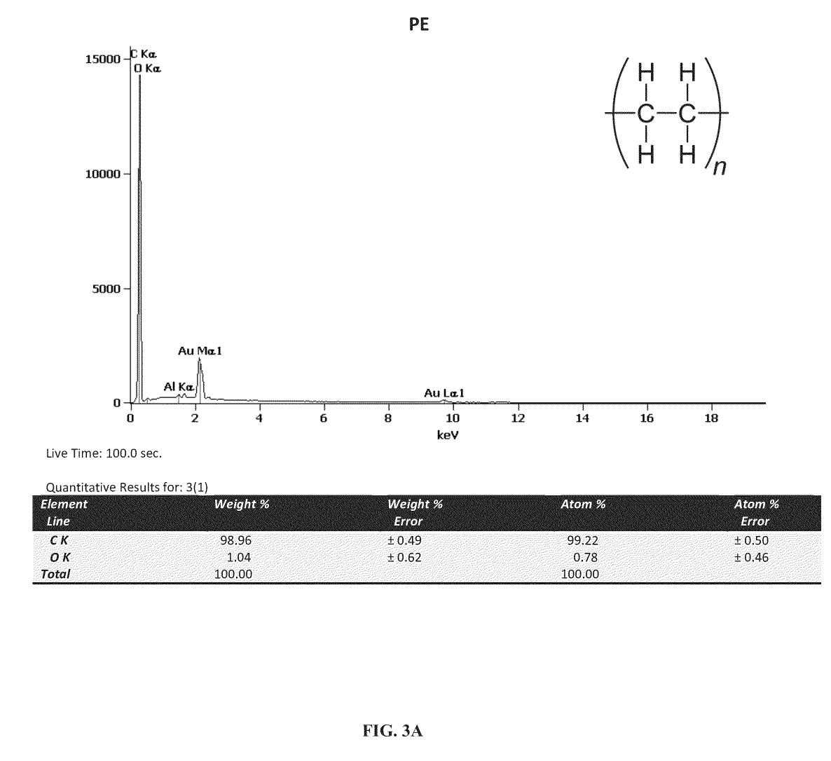

FIGS. 2A-C present SEM images of PE alone before extrusion (FIG. 2A) and of PE-RBS after co-extrusion (FIGS. 2B and 2C, cross-sectional and surface view, respectively).

As shown therein, the PE surface before extrusion is not totally smooth, having distinguished outstanding “pins” (FIG. 2A). After co-extrusion with RBS the surface structure of the polymer is similar to the intact PE both in cross-sectional (FIG. 2B) or surficial areas (FIG. 2B).

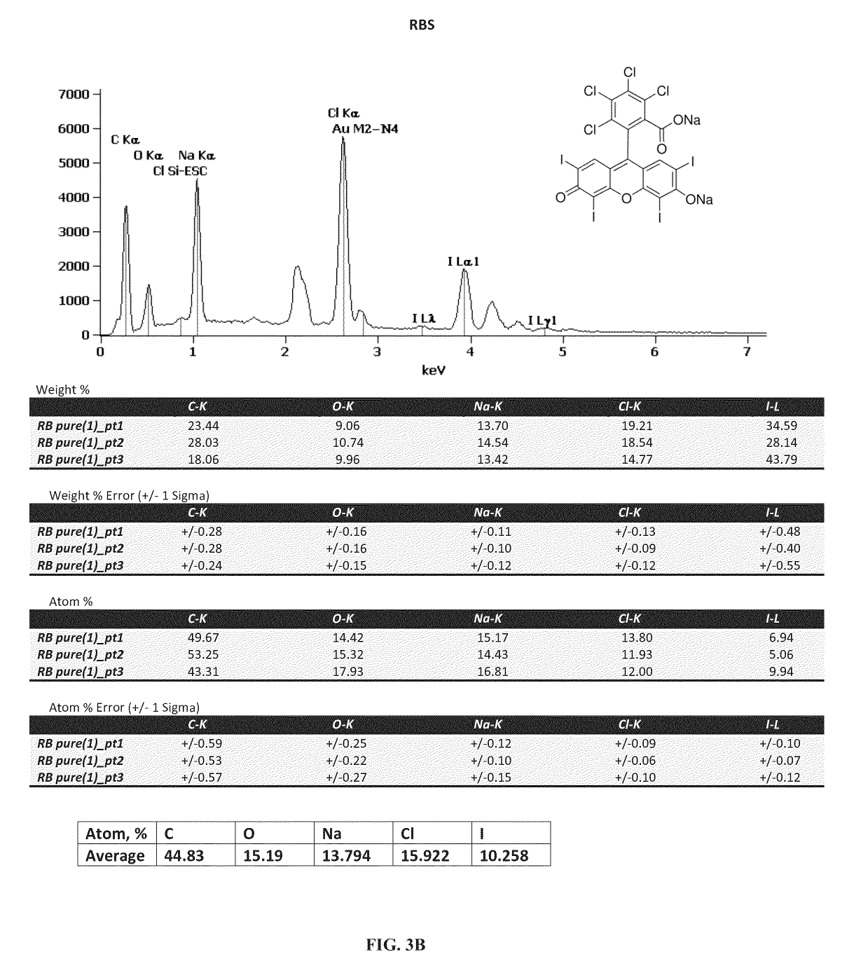

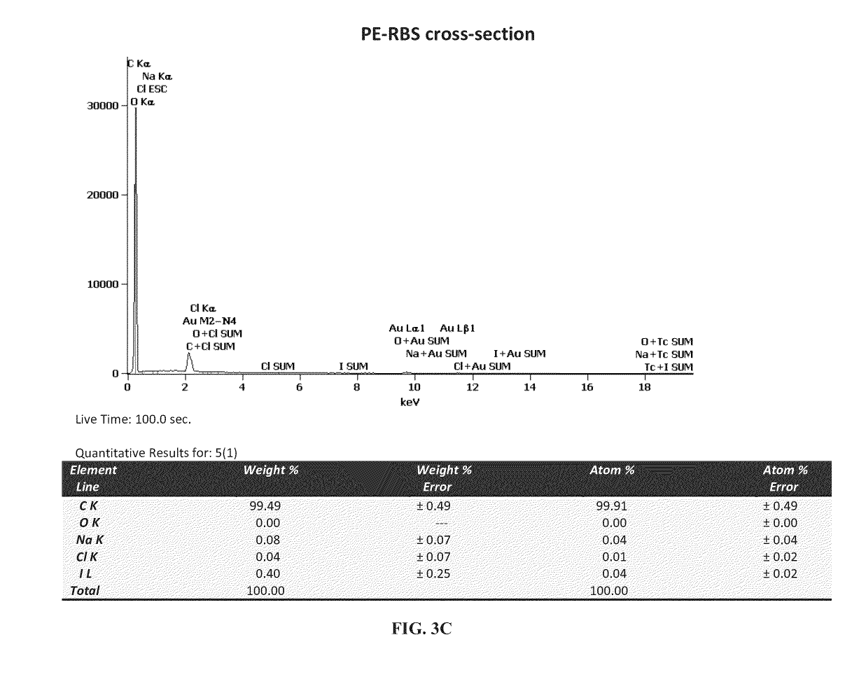

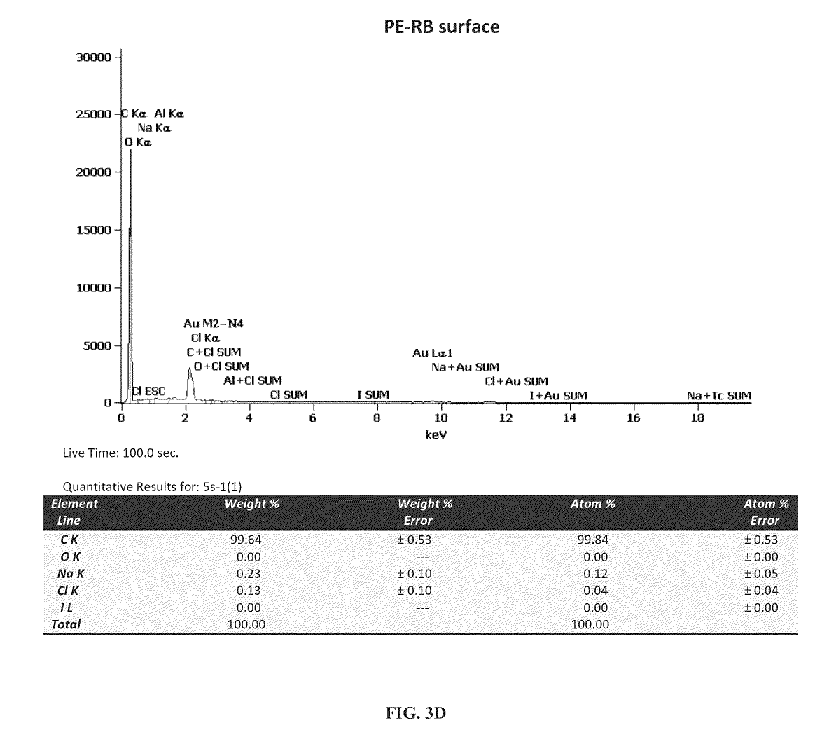

FIGS. 3A-D present results of EDS analyses of PE before extrusion (FIG. 3A), RBS alone (FIG. 3B), PE before extrusion (FIG. 3B) and PE-RBS after co-extrusion (FIGS. 3C and 3D showing the cross-section and surface, respectively). As shown therein, both PE and RBS separately show the presence of typical elements comprising same—PE is composed of carbon (hydrogen cannot be detected by the EDS method) (FIG. 3A) and RBS includes carbon, oxygen, sodium, chlorine and iodine (FIG. 3B). On the surface of the PE-RBS composition only carbon atoms are detected (FIG. 3C), indicating the absence of the RBS on the PE-RBS surface, and the presence of RBS molecules mostly within the polymeric matrix.

A Polymer Composition Comprising Polyethylene and Methylene Blue:

1000 grams of polyethylene beads (PE) and 10 grams of methylene blue chloride powder (MB) were mixed and coextruded in the extruder, as described hereinabove, to produce 5 mm diameter rods. The rods were chopped into 3 mm long pellets having a characteristic methylene blue color.

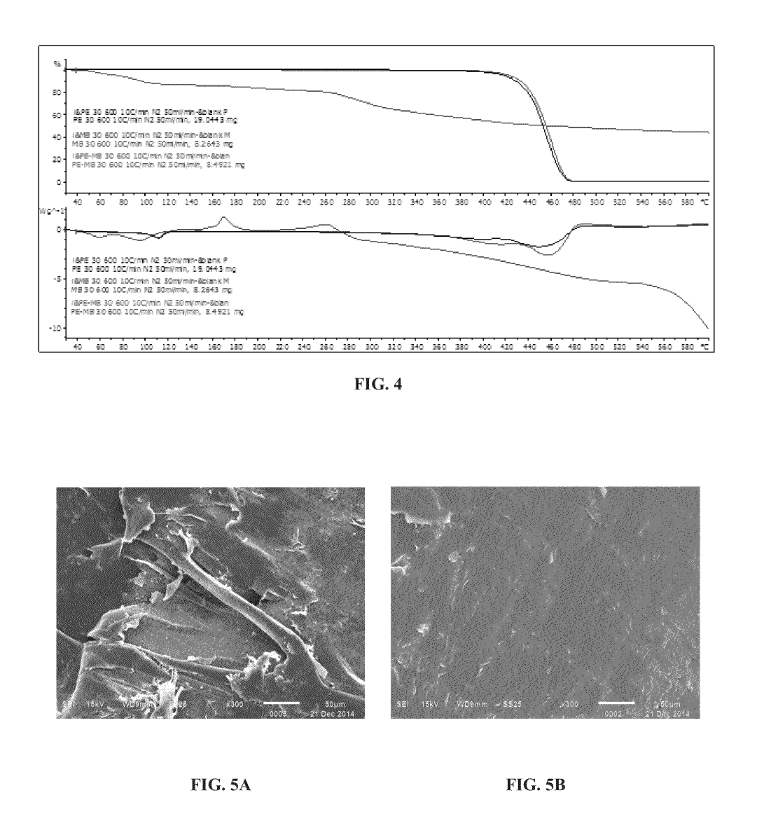

FIG. 4 presents the TGA data of PE (black), MB (blue) and PE-MB after co-extrusion (red). Upper diagram—mass changes and lower diagram—specific power changes.

As shown therein, MB alone has two decomposition regions—at 170° C. and 260° C., and PE alone decomposes at 450° C. PE-MB composition has two-step decomposition—at 410° C. and 458° C., with a main decomposition region at the latter temperature. No decomposition is seen at the temperatures characteristic for decomposition of the PS alone.

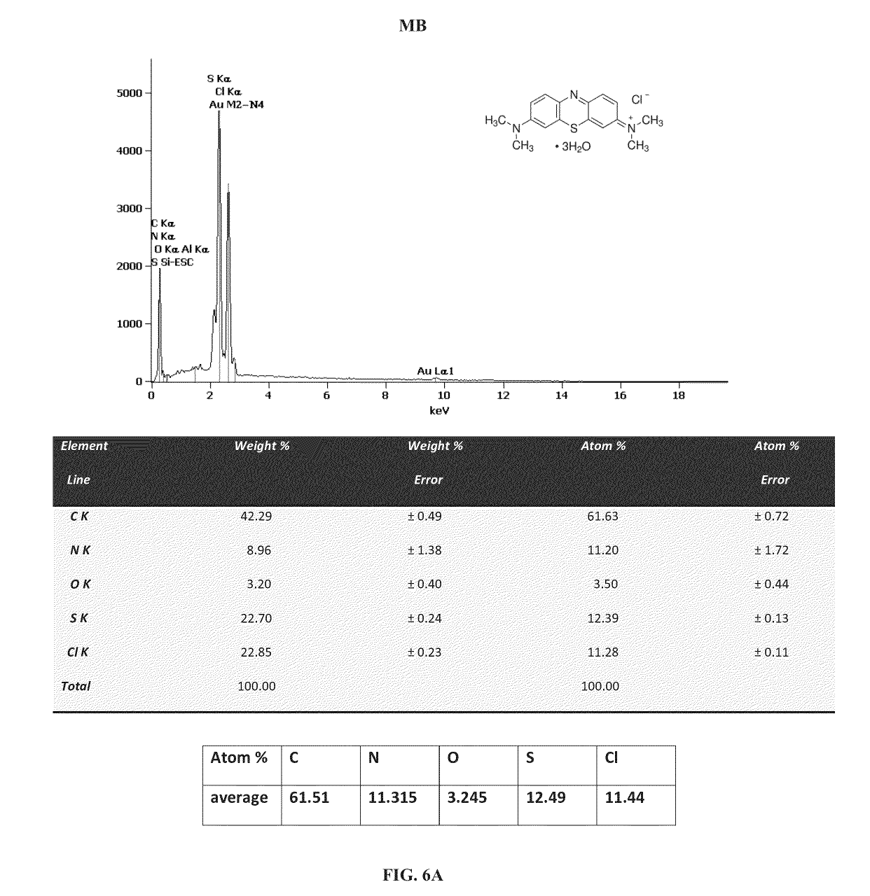

FIGS. 5A-B present SEM images of PE-MB after co-extrusion (FIGS. 5A and 5B, cross-sectional and external surface view, respectively).

As shown therein, surface view of the PE-MB cross-section is different from the external area, with the former being very rough (FIG. 5A) and not homogeneous and the latter being mostly smooth (FIG. 5B) and similar to PE alone (see, FIG. 2A).

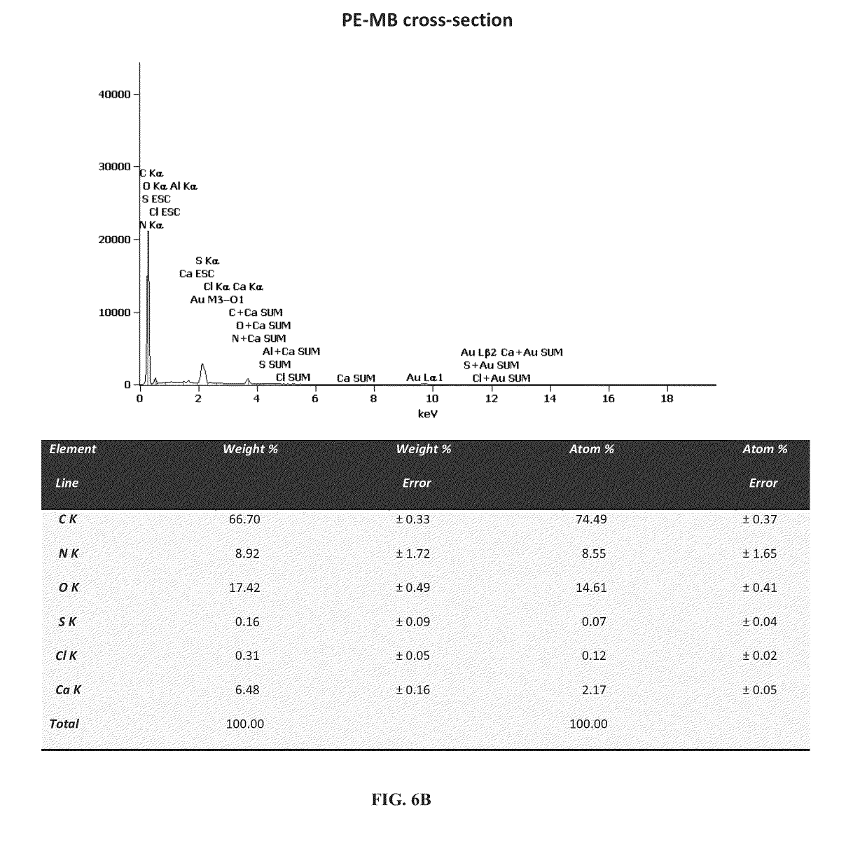

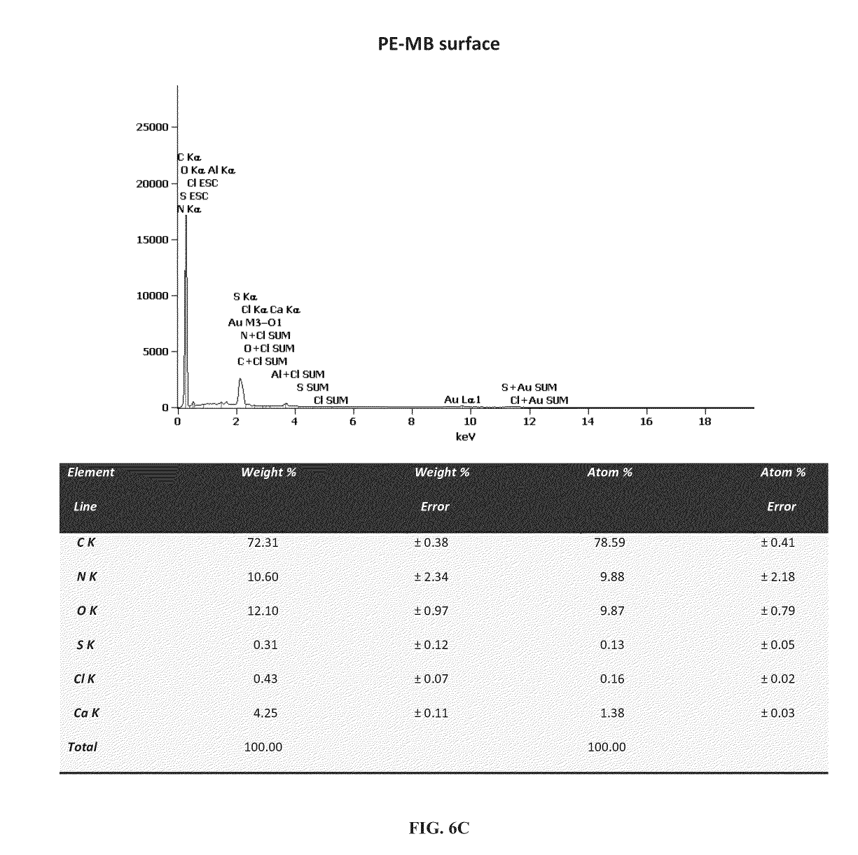

FIGS. 6A-C present EDS data of MB alone (FIG. 6A), and of the cross-section (FIG. 6B) and surface (FIG. 6C) of the PE-MB after co-extrusion.

As shown therein, element composition of MB alone reflects its molecular structure including carbon, nitrogen, sulfur, chlorine and oxygen when the origin of the latter lies in the presence of crystallohydrated water (FIG. 6A). Both cross-sectional (FIG. 6B) and external surface (FIG. 6C) areas exhibit the presence of nitrogen and oxygen, characteristic for MB, besides carbon which can be explained by non-homogeneous inclusion of MB into PE (which is not seen for PE-RBS).

A Polymer Composition Comprising Polypropylene and Rose Bengal:

1000 grams of polypropylene (PP) beads and 10 grams of Rose Bengal (RBS) powder were co-extruded in the extruder, as described hereinabove, to produce 5 mm diameter rods. The rods were chopped into 3 mm long pellets having a characteristic Rose Bengal color.

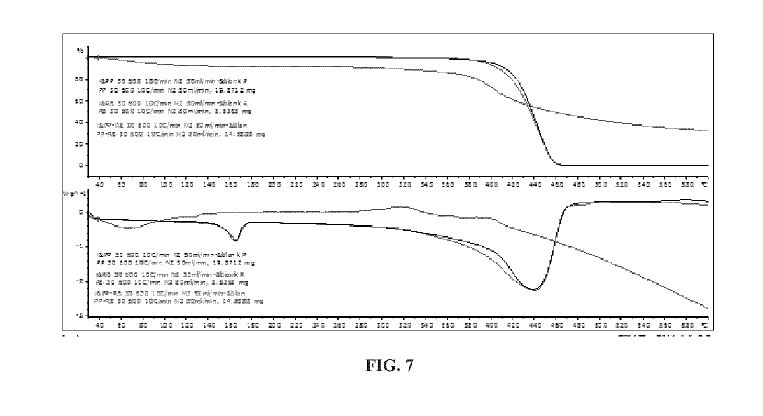

FIG. 7 presents the TGA data of PP (black), RBS (blue) and PP-RBS after co-extrusion (red). Upper diagram—mass changes and lower diagram—specific power changes. As can be seen, the thermal stability of PP-RB is very similar to that of PP alone; both polymers have the same decomposition temperature of 440° C.

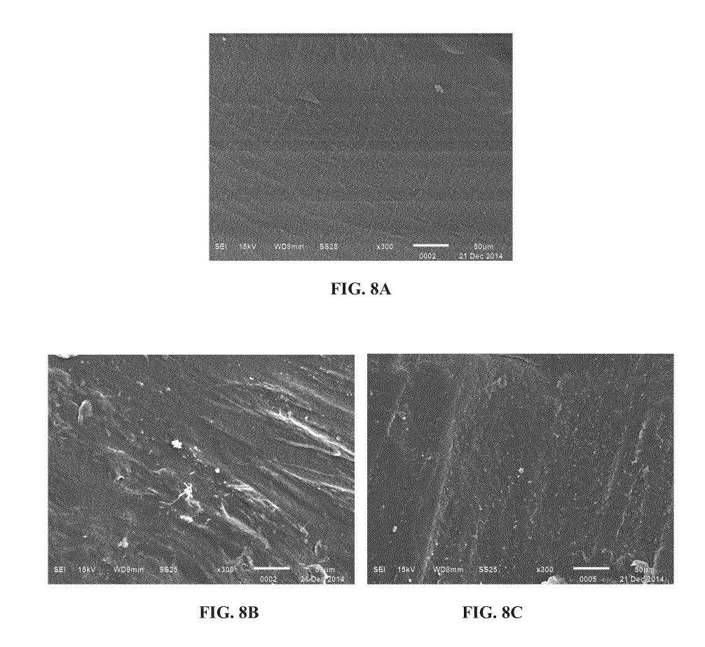

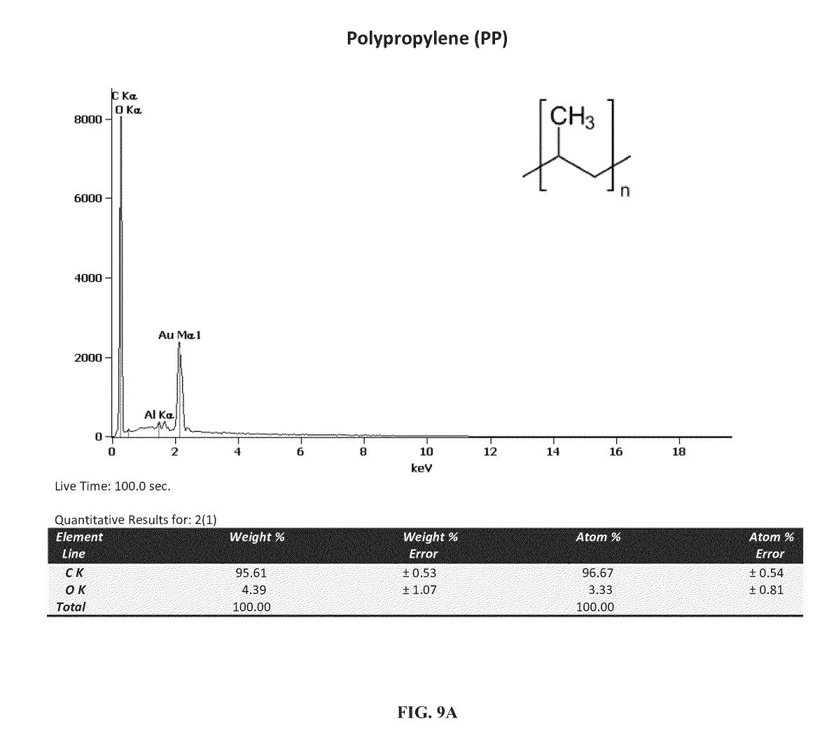

FIGS. 8A-C present SEM images of PP alone (FIG. 8A) and of PP-RBS after co-extrusion (FIGS. 8B and 8C, cross-sectional and surface view, respectively). As shown therein, the surface of both cross-sectional and external surficial areas of the PP-RBS (FIGS. 8B and 8C, respectively) are similar to the homogeneous surface of PP before the extrusion (FIG. 8A), although smoothness seems to be somewhat reduced.

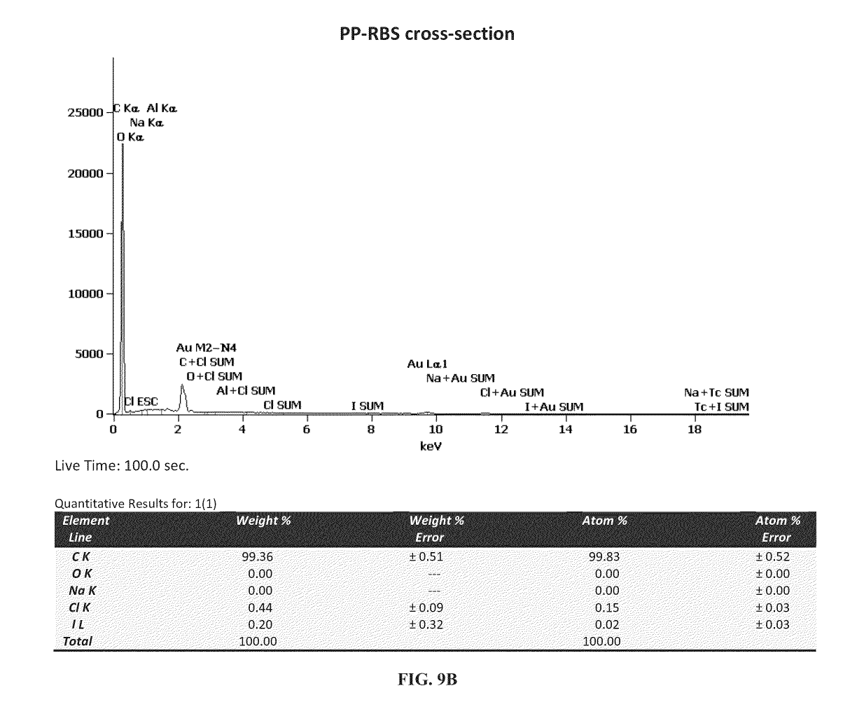

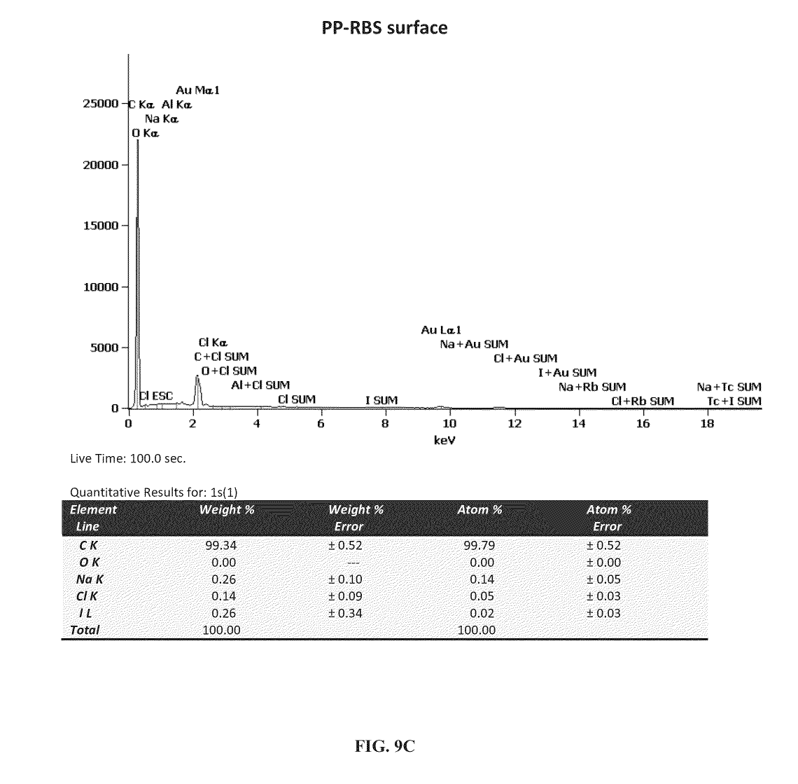

FIGS. 9A-C present EDS data of PP alone (FIG. 9A) and of the cross-section (FIG. 9B) and surface (FIG. 9C) of a PP-RBS composition after co-extrusion. In all cases, the only type of atoms which is detected on the polymer surfaces, is carbon, which means that molecules of RBS are not located on the polymer surface, but are incorporated into the PP matrix.

### Example 2

### Loading and Leaching Experiments

Loading and leaching of the PS were determined using UV spectroscopy, performed using a Cary 100bio UV-Visible Spectrophotometer, Varian, Australia.

Loading:

In an exemplary assay, after the co-extrusion of PE and RBS as described in Example 1, the inner chamber of the extruder was washed by water and absorbance of the washings was measured by a spectrometer at 557 nm. The amount of RBS which was not incorporated into the PE matrix was evaluated by multiplying the obtained absorbance by extinction coefficient and the volume of the washings.

Based on this assay, the amount of RBS incorporated in the PE polymeric matrix was determined to be 76% of the amount feeded to the extruder.

Similar assays are performed for the loading of RBL and MB into PE and of RBS into PP.

Leaching:

A sample of co-extruded polymer-PS pellets (200 grams) prepared as described in Example 1 hereinabove was soaked in a 2-Liter bath with tap water at room temperature for washing from non-entrapped PS. Water was changed 5 times, twice a day, and each time the washings were monitored by a spectrophotometer at 557 nm

The amount of leaked PS was evaluated by measuring the absorbance in the washing solutions, taking into account appropriate extinction coefficients and volumes of washings.

In the case of PE-RBS pellets, 0.8% of the incorporated RBS leaked from the polymer during the first 5 consecutive washings procedure and the rest RBS amount remained within the polymer structure. After this procedure the pellets were free from non-entrapped PS and no further leaching into the water was observed.

In the case of PE-MB and PE-RBL, residual PS leaching was observed after daily washings during one week.

### Example 3

### Antimicrobial Activity

Materials and Methods:

Bacterial Growth:

Cultures of Staphylococcus aureus (ATCC 25923), S. epidermidis (ATCC 12228); Streptococcus sp. (hospital isolate), P. aeruginosa (ATCC 25668) and Escherichia coli (ATCC 10798) were grown on brain-heart agar (BHA; Acumedia, USA) for 24 hours, and were thereafter transferred into brain-heart broth (BH; Acumedia) and grown at 37° C. and at a 170 rpm speed of shaking up to 3×10⁸ cells mL⁻¹ concentration. Cells were harvested by centrifugation, washed twice with 0.05 M phosphate-buffered saline (PBS), pH 6.5, diluted with PBS to a final concentration of 10⁸ cells mL⁻¹ and then were serially diluted in several (two to five) 10-fold dilutions.

Antibacterial Activity Assay:

The antibacterial activity of the polymer-PS compositions prepared as described in Example 1 hereinabove was studied in batch experiments as follows: 25 mL portions of a suspension of a bacterial cultures at a concentration of 10⁵ cells mL⁻¹ in sterile PBS were dispensed into Petri dishes with 5-8 grams of PE-RBS pellets. The plates were illuminated for 30-60 minutes with a white luminescent lamp emitting in the range of 400-700 nm and with an intensity of 1-2 mW cm⁻² (doses of 1.8-7.2 J cm⁻²). The light intensity was measured with a LX-102 Light-meter (Lutron, Taiwan). Control experiments were carried out with bacterial cultures on plates without the PE-RBS pellets, as well as in the presence of PE pellets not containing RBS, under illumination.

Antibacterial activity in continuous regime was studied by passing suspension of S. aureus in a saline solution from the top down a column (1×100 cm) packed with PE-RBS pellets. At the inlet and the outlet of the column samples were taken and bacterial concentration (CFU) was tested.

The number of colony forming units (CFU) was determined by the live count method.

The results obtained from at least three independent experiments carried out in duplicates were statistically analyzed by single-factor or two-factor ANOVA analyses. The difference between the results was considered significant if the P-value was less than 0.05.

### Experimental Results

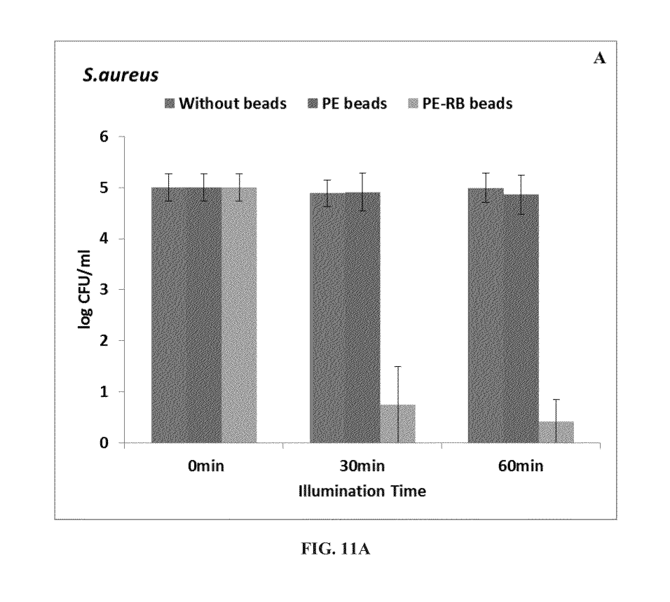

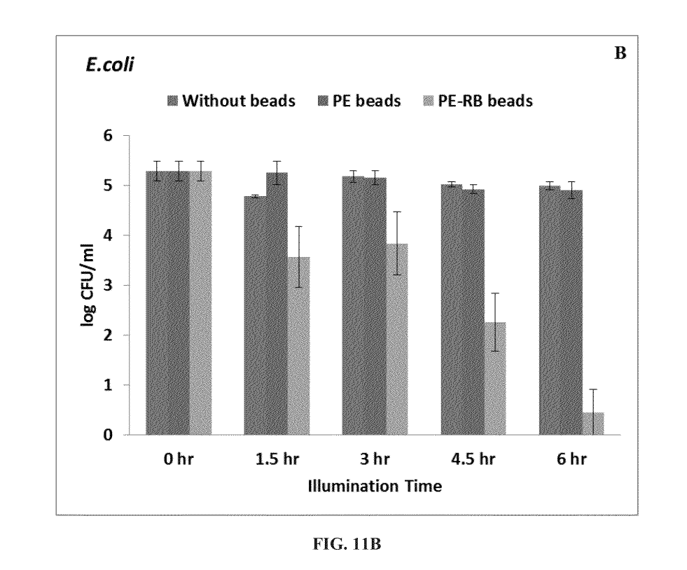

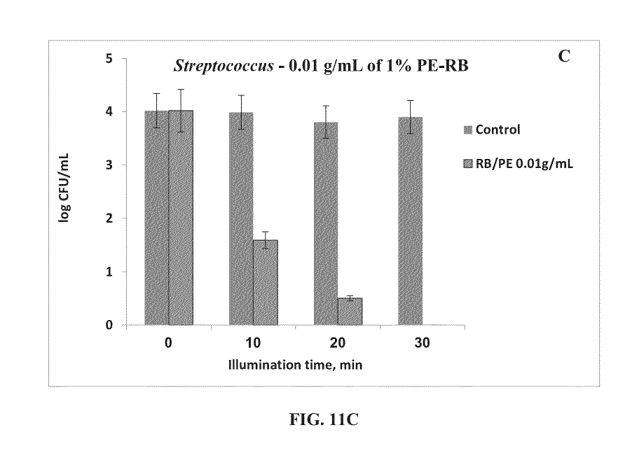

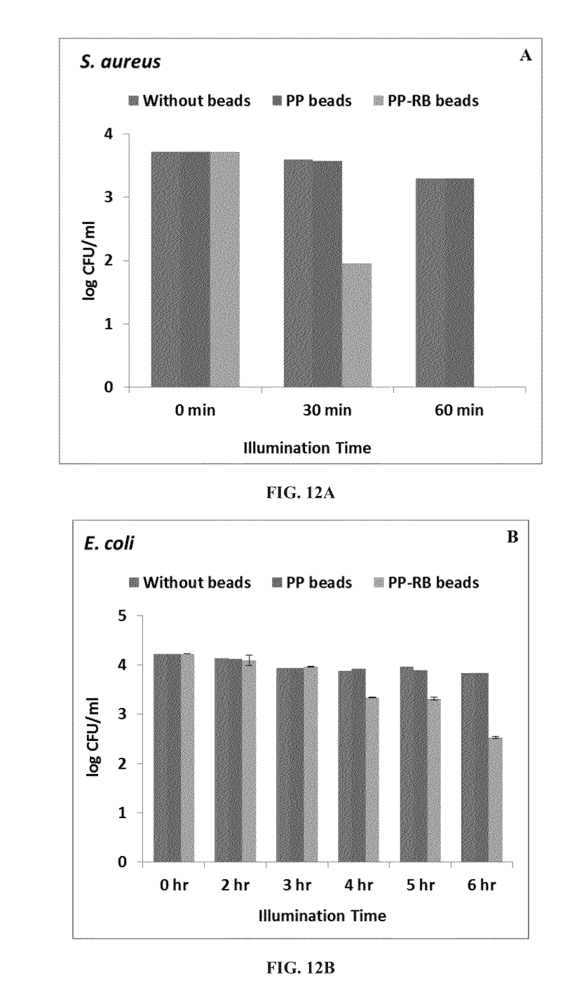

The results show that PE-RBS exhibits strong antibacterial activity (FIGS. 11A-C) and PP-RBS also possess antibacterial activity (FIGS. 12A-B).

PE-RBS eradicates more than 99.99% of S. aureus in 30 minutes and more than 99.997% in an hour of moderate illumination (FIG. 11A).

In the case of E. coli more than 99.5% of bacterial cells are inactivated after 4.5 hours and more than 99.998% are destroyed after 6 hours of illumination (FIG. 11B).

PE-RBS completely eradicated Streptococcus after 30 minutes of moderate illumination (FIG. 11C).

PP-RBS eradicates 98.3% of S. aureus after 30 minutes of illumination, and after an hour of illumination no bacterial cells are found (FIG. 12A).

In the case of E. coli, 98% of cells are inactivated after 6 hour illumination (FIG. 12B).

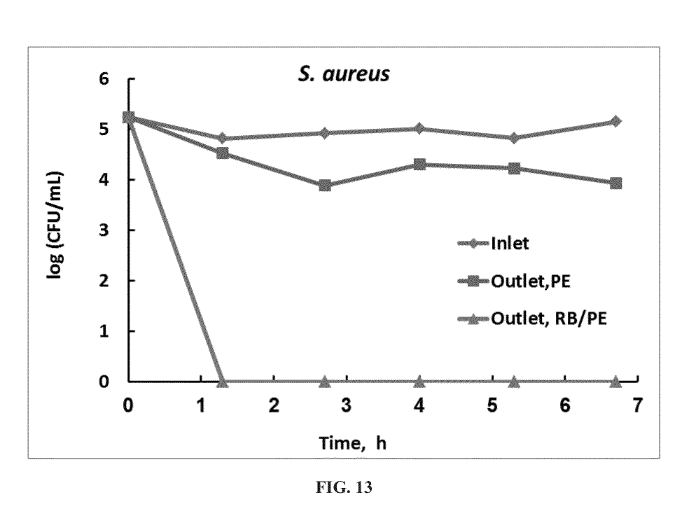

In the continuous regimen, after about 80 minutes the system reached an equilibrium state and from this moment no bacterial cells were revealed at the outlet from the column (FIG. 13). At the same time at the outlet from the control column packed with PE beads bacterial concentration hardly differed from that tested at the inlet of the column.

In additional experiments, using the same protocol as described hereinabove, polyethylene (PE) beads loaded with 1% RBS or MB were used at various bead concentrations (from 0.01 to 0.32 g/mL) and applied against S. aureus.

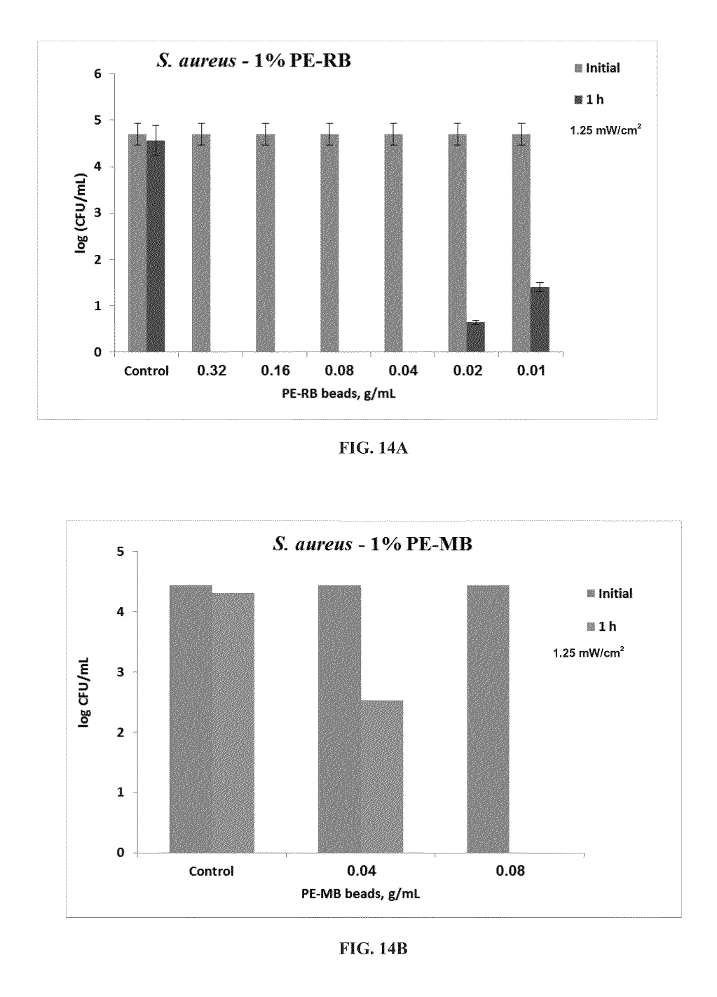

After 1 hour of illumination at light intensity of 1.25 mW/cm² bacterial cells were totally eradicated when PE-RBS fraction was 0.04 g/mL (FIG. 14A) and PE-MB fraction was 0.08 gram/mL (FIG. 14B).

The effect of the beads was also tested at various concentrations of the bacterial cultures.

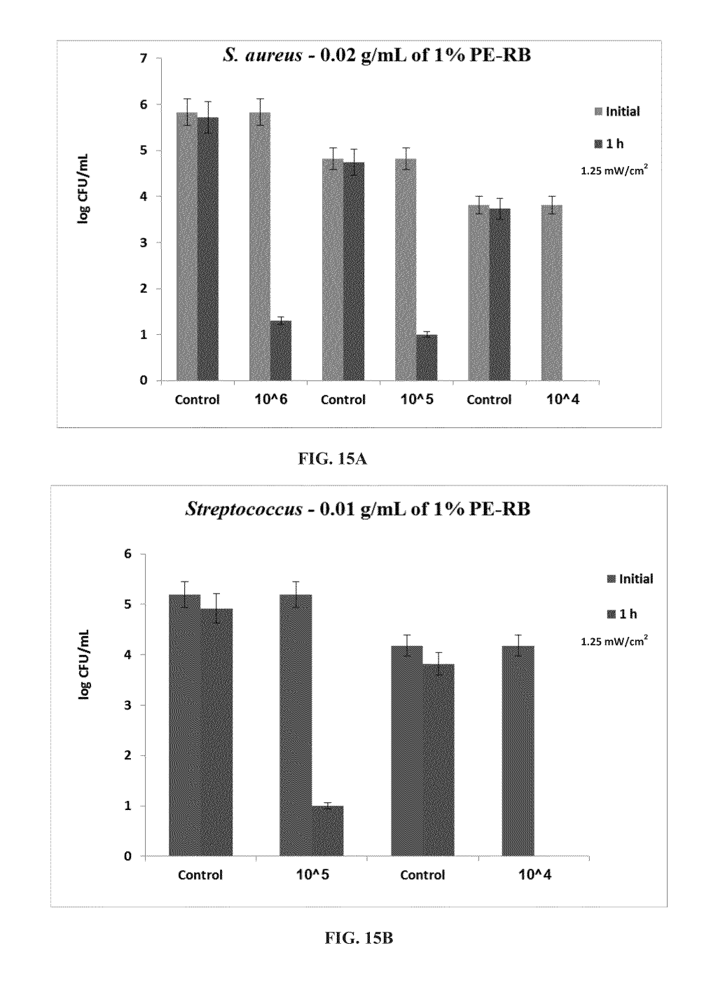

For Gram-positive S. aureus (FIG. 15A) and Streptococcus (FIG. 15B) total cell inhibition was achieved at initial cell concentration of 10⁴ CFU/mL after 1 hour of illumination. At initial cell concentration of 10⁵ CFU/mL the rate of inhibition was 99.99% after the same time for both bacteria.

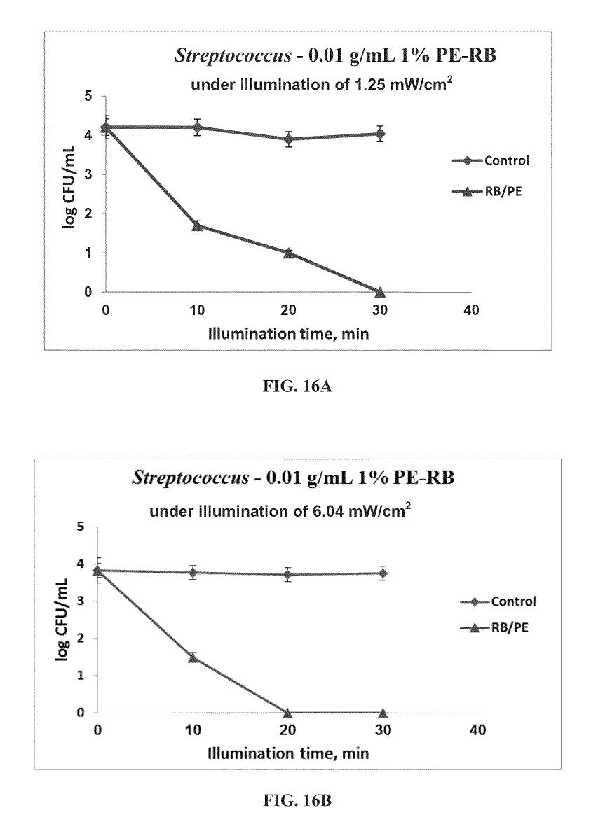

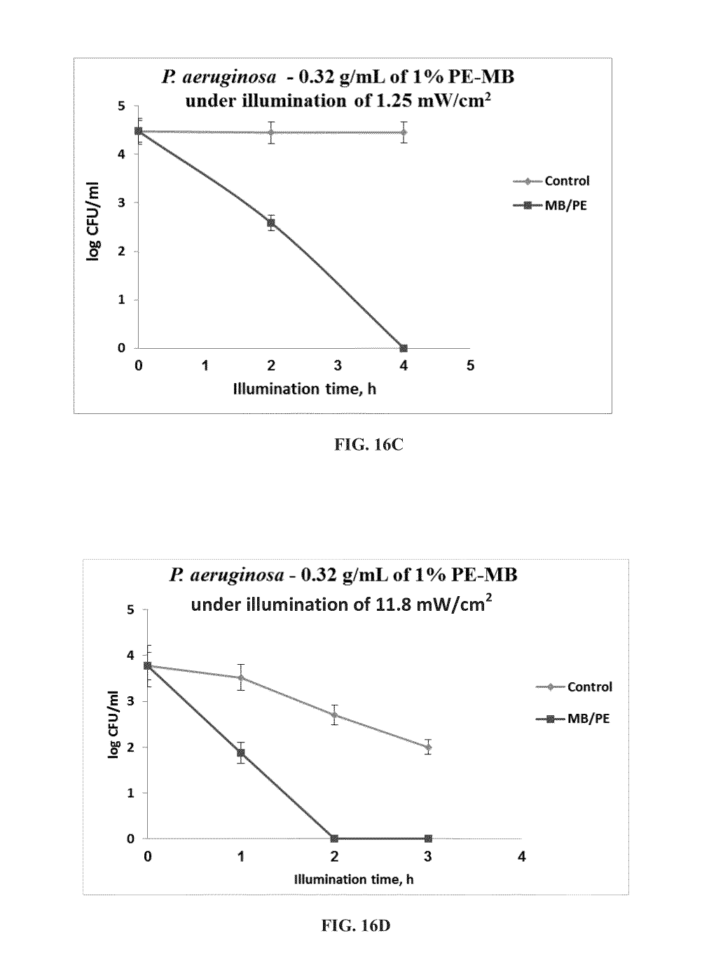

The effect of illumination on the activity of the PS beads was tested for Gram-positive Streptococcus and Gram-negative P. aeruginosa. Beads of PE-RBS added at a concentration of 0.01 g/mL to planktonic cells of Streptococcus caused total eradication of bacteria after 30 minutes of treatment under illumination of 1.25 mW/cm² (FIG. 16A). Increase in illumination intensity to 6.04 mW/cm² leads to reduction in time of cell inhibition to 20 minutes (FIG. 16B).

In the case of P. aeruginosa, PE-MB beads killed cells after 4 hours of illumination at 1.25 mW/cm² (FIG. 16C) and after only 2 hours when illuminated at 11.8 mW/cm² (FIG. 16D).

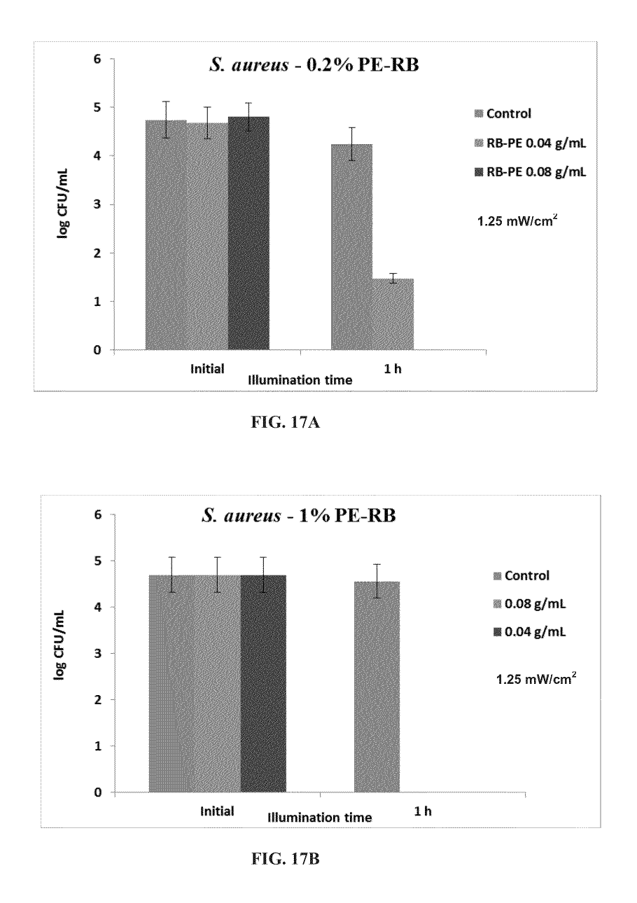

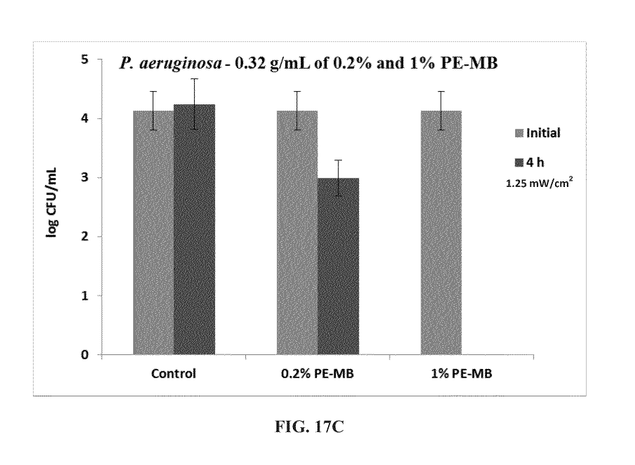

PS beads were also tested as various loading of the PS in the polymeric matrix. Polyethylene beads carrying 0.2% of RBS totally eradicated cells of S. aureus at bead fraction of 0.08 g/mL (FIG. 17A). At 1% loading of RBS total cell eradication occurred already at bead fraction of 0.04 g/mL (FIG. 17B).

Similar tendency was seen for P. aeruginosa cells treated by PE-MB. At 0.2% loading of MB partial reduction of cells was observed, and at 1% loading of MB in the beads cells were totally eradicated (FIG. 17C).

### Example 4

Photostability Photostability experiments were performed by placing 14 grams of PP-RBS into a 3-liter bath with tap water into which suspensions of S. aureus bacteria were added daily. The control bath did not any polymeric material and any PS. Both baths were permanently illuminated by a luminescent lamp. Samples from both baths were taken daily before addition of fresh portions of bacteria and tested for live bacteria concentration.

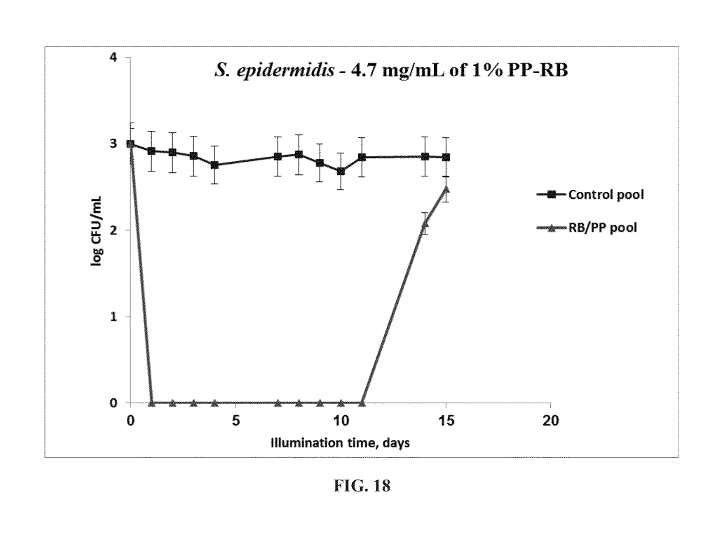

The results are presented in FIG. 18 and show that for at least 11 days the antibacterial stability of PP-RBS remained unchanged, and cells of S. epidermidis were completely eradicated. On the day 14, a partial deactivation was observed, presumably due to photobleaching, yet, more than 80% of the cells were eradicated. No substantial change in cells count was observed in the control group.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

What is claimed is:

1. A composition comprising a polymeric matrix and a photosensitizer incorporated in said polymeric matrix, the polymeric matrix comprising a thermoplastic polymer, the composition being devoid of a surfactant and/or a residual amount of an organic solvent.

2. The composition of claim 1, wherein the polymer matrix consists essentially of said thermoplastic polymer and said photosensitizer.

3. The composition of claim 1, consisting essentially of said thermoplastic polymer and said photosensitizer.

4. The composition of claim 1, wherein said thermoplastic polymer has a Tm value lower than a temperature at which said photosensitizer decomposes.

5. The composition of claim 1, wherein said thermoplastic polymer is a non-soluble thermoplastic polymer.

6. The composition of claim 5, wherein said non-soluble thermoplastic polymer is a polyolefin.

7. The composition of claim 1, wherein said photosensitizer is selected from the group consisting of a xanthene dye, a porphyrin, a phenothiazine dye, a triphenylmethine dye, an oxazine dye, a phthalocyanine, a psoralene and a perylenequinonoid.

8. The composition of claim 1, wherein said photosensitizer is xanthene dye.

9. The composition of claim 1, wherein an amount of said one or more photosensitizers is in the range of from about 0.1 weight percent to about 10 weight percents of the total weight of the composition-of-matter.

10. The composition of claim 1, wherein less than 20% of said photosensitizer is present on an external surface of said polymeric matrix.

11. A process of preparing the composition of claim 1, the process comprising:
heating a solid mixture comprising said photosensitizer and said thermoplastic polymer at a temperature equal to or higher than a Tm of said polymer, to thereby obtain a fluid mixture of said photosensitizer and said polymer; and
cooling said fluid mixture to a temperature below said Tm of said polymer, thereby obtaining the composition.

12. The process of claim 11, wherein said solid mixture and said fluid mixture are devoid of an organic solvent and/or a surfactant.

13. The process of claim 11, wherein said solid mixture and said fluid mixture consist essentially of said polymer and said photosensitizer.

14. The process of claim 11, wherein a weight ratio of said polymer and said photosensitizer in said solid mixture ranges from 1000:1 to 10:1.

15. A process of preparing an antimicrobial composition, the process comprising heating a solid mixture comprising a thermoplastic polymer and a photosensitizer at a temperature equal to or higher than a Tm of said polymer, to thereby obtain a fluid mixture of said photosensitizer and said polymer; and cooling said fluid mixture, to thereby obtain the antimicrobial composition.

16. The process of claim 15, wherein said Tm of the said polymer is lower than a temperature at which said photosensitizer decomposes.

17. The process of claim 15, wherein said solid mixture and said fluid mixture are devoid of an organic solvent and/or a surfactant.

18. The process of claim 15, wherein said solid mixture and said fluid mixture consist essentially of said polymer and said photosensitizer.

19. The process of claim 15, wherein a weight ratio of said polymer and said photosensitizer in said solid mixture ranges from 1000:1 to 10:1.

20. The process of claim 15, said thermoplastic polymer is selected from the group consisting of a polyolefin, a polyester, a polycarbonate, a polyurethane, a polyether urethane, a polyether carbonate, a polyester carbonate, a polyester urethane, a polyanhydride, a polyamide, a polyacrylate, a polymethacrylate, and any copolymer of the forgoing.

21. The process of claim 15, wherein said thermoplastic polymer is a non-soluble thermoplastic polymer.

22. The process of claim 15, wherein said photosensitizer is selected from the group consisting of a xanthene dye, a porphyrin, a phenothiazine dye, a triphenylmethine dye, an oxazine dye, a phthalocyanine, a psoralene and a perylenequinonoid.

23. An antimicrobial composition prepared by the process of claim 15.

24. The antimicrobial composition of claim 23, being devoid of a surfactant and/or a residual amount of an organic solvent.

25. The composition of claim 23, wherein an amount of said one or more photosensitizers is in the range of from about 0.1 weight percent to about 10 weight percents of the total weight of the composition.

26. The composition of claim 23, wherein less than 20% of said photosensitizer is present on an external surface of said polymeric matrix.

27. An article-of-manufacturing comprising the composition of claim 1.

28. An article-of-manufacturing comprising the composition of claim 23.

29. A method of disinfecting or sterilizing the composition of claim 1, the method comprising exposing the composition or the article-of-manufacturing to conditions in which said photosensitizer generates reactive oxygen species.

30. A method of disinfecting or sterilizing the composition of claim 23, the method comprising exposing the composition or the article-of-manufacturing to conditions in which said photosensitizer generates reactive oxygen species.

31. A method of disinfecting or sterilizing the article-of-manufacture of claim 27, the method comprising exposing the composition or the article-of-manufacturing to conditions in which said photosensitizer generates reactive oxygen species.

32. A method of disinfecting or sterilizing the article-of-manufacture of claim 28, the method comprising exposing the composition or the article-of-manufacturing to conditions in which said photosensitizer generates reactive oxygen species.

33. A method of disinfecting a substrate, the method comprising contacting the substrate with the composition of claim 1, or with an article-of-manufacturing comprising said composition, and exposing the composition or the article-of-manufacturing to conditions in which said photosensitizer generates reactive oxygen species.

34. The method of claim 33, wherein said substrate is a bodily tissue, fluid or cavity, the method being for treating a medical condition associated with a microorganism in a subject in need thereof.

35. The method of claim 33, wherein said substrate is an inanimate substrate.

36. The method of claim 35, wherein said substrate comprises water.

37. The method of claim 36, being for disinfecting said water.

38. A method of disinfecting a substrate, the method comprising contacting the substrate with the composition of claim 23 or with an article-of-manufacturing comprising said composition, and exposing the composition or the article-of-manufacturing to conditions in which said photosensitizer generates reactive oxygen species.

39. The method of claim 38, wherein said substrate is a bodily tissue, fluid or cavity, the being for treating a medical condition associated with a microorganism in a subject in need thereof.

40. The method of claim 38, wherein said substrate comprises water.

41. The method of claim 40, being for disinfecting said water.

42. A method of disinfecting a substrate, the method comprising contacting the substrate with the composition of claim 23, and exposing the composition or the article-of-manufacturing to conditions in which said photosensitizer generates reactive oxygen species.

43. The method of claim 42, wherein said substrate is a bodily tissue, fluid or cavity, the being for treating a medical condition associated with a microorganism in a subject in need thereof.

44. The method of claim 42, wherein said substrate comprises water.

45. The method of claim 44, being for disinfecting said water.
